(19) United States
(12) Patent Application Publication
(10) Pub. No.: US 2018/0299473 A1
(43) Pub. Date: Oct. 18, 2018

(54) Methods and Systems for Point-of-Care Sample Analysis

(71) Applicant: Essenlix Corp., Monmouth Junction, NJ (US)

(72) Inventor: Stephen Y. Chou, Princeton, NJ (US)

(21) Appl. No.: 15/763,796

(22) PCT Filed: Sep. 27, 2016
(86) PCT No.: PCT/US16/53999

§ 371 Date: Mar. 27, 2018

Related U.S. Application Data
(60) Provisional application No. 62/233,885, filed on Sep. 28, 2015.

(51) Int. Cl. G01N 35/00; B01L 3/00; G01N 33/53; G01N 35/08; G01N 33/487

(52) U.S. Cl. CPC G01N 35/00871; B01L 3/5027; G06F 19/00; G01N 35/08; G01N 33/487; G01N 33/53

(57) ABSTRACT

Provided herein is a method of analyzing a biological sample collected from a subject. An implementation of the method may include a) receiving, at a first site, data transmitted from a data acquisition device at a second site, wherein the data acquisition device is configured to i) read an output signal that is representative of the biological sample from a disk-coupled dots-on-pillar antenna array (D2PA) device comprising a D2PA to generate raw data that is representative of the biological sample, and ii) transmit data comprising the raw data to the first site, and b) analyzing the data at the first site to generate an evaluation of the data. In some embodiments, the method includes providing a process management operation such that the evaluation can be used for screening, diagnosis, or treatment of the subject. Also provided herein are systems that fmd use in implementing the method.

### CROSS-REFERENCING

This application claims the benefit of provisional application Ser. No. 62/233,885, filed on Sep. 28, 2015, which is incorporated herein in its entirety for all purposes.

### BACKGROUND

Traditional ways of collecting and analyzing patient-derived samples in a centralized laboratory suffers from a number of issues, such as sample degradation, variability in sample quality and risk of sample mishandling. Point-of-care testing provides a number of advantages over traditional methods by obviating the need for sample transport and reducing the number of handling steps before analysis. Despite its advantages, a point-of-care sample analysis system still requires sophisticated equipment, such as bulky and costly assay readers, and requires the use of intrusive processes to collect sample, such as blood, from the patient.

### SUMMARY

Provided herein is a method of analyzing a biological sample collected from a subject. An implementation of the method may include a) receiving, at a first site, data transmitted from a data acquisition device at a second site, wherein the data acquisition device is configured to i) read an output signal that is representative of the biological sample from a disk-coupled dots-on-pillar antenna array (D2PA) device comprising a D2PA to generate raw data that is representative of the biological sample, and ii) transmit data comprising the raw data to the first site, and b) analyzing the data at the first site to generate an evaluation of the data.

In any embodiment, the data acquisition device may be a mobile device, such as a mobile phone, a smart phone, a tablet, or a laptop.

In some embodiments, the first site and the second site are the same. In other embodiments, the first site is remote from the second site.

In any embodiment, the method may include generating a report comprising the evaluation of the data. In some embodiments, the method further includes transmitting the report from the first remote site to a report display device, wherein the report display device is configured to receive and display the report. In some embodiments, the report display device is at a second site that is remote from the first site. In some embodiments, the report display device is the same device as the data acquisition device.

In any embodiment, the output signal may include luminescence. In some embodiments the raw data is luminescence data that is representative of the sample.

In any embodiment, the raw data may be representative of the amount of the analyte in the sample.

In any embodiment, the data acquisition device may be configured to read an output signal from the sample acquisition device by acquiring an image of the D2PA to generate raw data.

In any embodiment, the D2PA device may be a microfluidic device or a micro titer plate.

In any embodiment, the D2PA may include a binding agent that specifically binds to the analyte of interest. In some embodiments, the binding agent is an antibody or a nucleic acid.

In any embodiment, the first site may include an authorized analytical facility and the D2PA device may be configured to transmit the data to the authorized analytical facility. In some embodiments, the authorized analytical facility is a Clinical Laboratory Improvement Amendments (CLIA)-compliant laboratory.

In any embodiment, the second site may be the subject's home, a health assessment location, or a health treatment location.

In any embodiment, the evaluation may be used by a health care professional for screening, diagnosis, or treatment of the subject. In some embodiments, the evaluation can be used by the subject for diagnosis of the subject.

In any embodiment, the method further may include c) providing a process management operation such that the evaluation can be used for screening, diagnosis, or treatment of the subject. In some embodiments, the process management operation includes one or more of i) quality assurance of the raw data, ii) quality assurance of the data acquisition device and/or D2PA device, iii) oversight of the use of the data acquisition device and/or D2PA device, iv) oversight of the analysis performed on the data to generate the evaluation, and v) authorization of access to the evaluation and/or data by the health care professional.

Also provided herein is a method of analyzing a biological sample, including a) transmitting to a first site, using a data acquisition device at a second site, data comprising raw data that is representative of a biological sample collected from a subject, wherein the raw data includes a readout from a D2PA device comprising a D2PA, wherein the readout is obtained by reading an output signal from the D2PA device with the data acquisition device, wherein the D2PA device is configured to receive the biological sample, and contact the received sample with the D2PA, wherein the D2PA is configured to bind an analyte of interest, and generate the output signal upon contacting the biological sample, wherein the output signal is representative of the sample, wherein the first site comprises a communication device configured to receive data transmitted from the data acquisition device, and a processor configured to analyze and generate an evaluation of the data a communication device that receives data transmitted from the data acquisition device, and a processor that analyzes and generates an evaluation of the data, and b) receiving a report comprising an evaluation of the data. In some embodiments, the raw data is representative of the amount of the analyte in the sample. In some embodiments, the evaluation of the data includes the amount of the analyte of interest in the sample, and a range of values for the analyte in an individual free of or at low risk of being diagnosed with a disease or a health condition, wherein the amount of the analyte relative to the range of values is diagnostic of the disease or health condition.

In any embodiment, the evaluation of the data includes a diagnosis for a disease or a health condition provided by a health care professional.

In any embodiment, the evaluation of the data includes a recommendation to seek medical attention.

In any embodiment, the processor may provide a process management operation such that the evaluation can be used for screening, diagnosis, or treatment of the subject.

Also provided herein are systems that find use in implementing the present method.

### BRIEF DESCRIPTION OF THE FIGURES

The skilled artisan will understand that the drawings, described below, are for illustration purposes only. The drawings are not intended to limit the scope of the present teachings in any way.

FIG. 1 shows a schematic diagram of an embodiment of the present disclosure.

FIG. 2 shows a schematic diagram of an embodiment of the present disclosure.

FIG. 3 is a drawing showing acquisition of data from a disk-coupled dots-on-pillar array (D2PA) device using a mobile device, according to an embodiment of the present disclosure.

FIG. 4 is an image and drawing of a D2PA device, according to an embodiment of the present disclosure.

FIG. 5 is a collection of images and a graph showing detection of an analyte on D2PA, according to an embodiment of the present disclosure.

FIG. 6 is a collection of images and a schematic diagram showing a mobile device-based point-of-care testing device, according to an embodiment of the present disclosure.

### DEFINITIONS

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The terms “polynucleotide”, “nucleotide”, “nucleotide sequence”, “nucleic acid”, “nucleic acid molecule”, “nucleic acid sequence” and “oligonucleotide” are used interchangeably, and can also include plurals of each respectively depending on the context in which the terms are utilized. They refer to a polymeric form of nucleotides of any length, either deoxyribonucleotides (DNA) or ribonucleotides (RNA), or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA, ribozymes, small interfering RNA, (siRNA), microRNA (miRNA), small nuclear RNA (snRNA), cDNA, recombinant polynucleotides, branched polynucleotides, plasmids, vectors, isolated DNA (A, B and Z structures) of any sequence, PNA, locked nucleic acid (LNA), TNA (treose nucleic acid), isolated RNA of any sequence, nucleic acid probes, and primers. LNA, often referred to as inaccessible RNA, is a modified RNA nucleotide. The ribose moiety of an LNA nucleotide is modified with an extra bridge connecting the 2′ and 4′ carbons. The bridge “locks” the ribose in the 3′-endo structural conformation, which is often found in the A-form of DNA or RNA, which can significantly improve thermal stability.

A “capture agent” as used herein, refers to a binding member, e.g. nucleic acid molecule, polypeptide molecule, or any other molecule or compound, that can specifically bind to its binding partner, e.g., a second nucleic acid molecule containing nucleotide sequences complementary to a first nucleic acid molecule, an antibody that specifically recognizes an antigen, an antigen specifically recognized by an antibody, a nucleic acid aptamer that can specifically bind to a target molecule, etc. A capture agent may concentrate the target molecule from a heterogeneous mixture of different molecules by specifically binding to the target molecule. Binding may be non-covalent or covalent. The affinity between a binding member and its binding partner to which it specifically binds when they are specifically bound to each other in a binding complex is characterized by a K_{D} (dissociation constant) of 10⁻⁵ M or less, 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, including 10⁻¹⁶ M or less. “Affinity” refers to the strength of binding, increased binding affinity being correlated with a lower K_{D}.

The term “a secondary capture agent” which can also be referred to as a “detection agent” refers a group of biomolecules or chemical compounds that have highly specific affinity to the antigen. The secondary capture agent can be strongly linked to an optical detectable label, e.g., enzyme, fluorescence label, or can itself be detected by another detection agent that is linked to an optical detectable label through bioconjugation (Hermanson, “Bioconjugate Techniques” Academic Press, 2nd Ed., 2008).

By “antibody,” as used herein, is meant a protein consisting of one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (μ), delta (δ), gamma (γ), sigma (σ), and alpha (α) which encode the IgM, IgD, IgG, IgE, and IgA antibody “isotypes” or “classes” respectively. Antibody herein is meant to include full length antibodies and antibody fragments, and may refer to a natural antibody from any organism, an engineered antibody, or an antibody generated recombinantly for experimental, therapeutic, or other purposes. The term “antibody” includes full length antibodies, and antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies.

The terms “antibody epitope,” “epitope,” “antigen” are used interchangeably herein to refer to a biomolecule that is bound by an antibody. Antibody epitopes can include proteins, carbohydrates, nucleic acids, hormones, receptors, tumor markers, and the like, and mixtures thereof. An antibody epitope can also be a group of antibody epitopes, such as a particular fraction of proteins eluted from a size exclusion chromatography column. Still further, an antibody epitope can also be identified as a designated clone from an expression library or a random epitope library.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these.

As is known to one skilled in the art, hybridization can be performed under conditions of various stringency. Suitable hybridization conditions are such that the recognition interaction between a capture sequence and a target nucleic acid is both sufficiently specific and sufficiently stable. Conditions that increase the stringency of a hybridization reaction are widely known and published in the art. See, for example, Green, et al., (2012), infra.

“Conditions suitable for binding,” as used herein with respect to binding of a capture agent to an analyte, e.g., a biomarker, a biomolecule, a synthetic organic compound, an inorganic compound, etc., refers to conditions that produce nucleic acid duplexes, protein/protein (e.g., antibody/antigen) complexes, protein/compound complexes, aptamer/target complexes that contain pairs of molecules that specifically bind to one another, while, at the same time, disfavor the formation of complexes between molecules that do not specifically bind to one another. Specific binding conditions are the summation or combination (totality) of both hybridization and wash conditions, and may include a wash and blocking steps, if necessary.

For nucleic acid hybridization, specific binding conditions can be achieved by incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, and 20 μg/ml denatured, sheared salmon sperm DNA, followed by washing the filters in 0.1×SSC at about 65° C.

For binding of an antibody to an antigen, specific binding conditions can be achieved by blocking a substrate containing antibodies in blocking solution (e.g., PBS with 3% BSA or non-fat milk), followed by incubation with a sample containing analytes in diluted blocking buffer. After this incubation, the substrate is washed in washing solution (e.g. PBS+TWEEN 20) and incubated with a secondary capture antibody (detection antibody, which recognizes a second site in the antigen). The secondary capture antibody may conjugated with an optical detectable label, e.g., a fluorophore such as IRDye800CW, Alexa 790, Dylight 800. After another wash, the presence of the bound secondary capture antibody may be detected. One of skill in the art would be knowledgeable as to the parameters that can be modified to increase the signal detected and to reduce the background noise.

A “plurality” contains at least 2 members. In certain cases, a plurality may have at least 10, at least 100, at least 1000, at least 10,000, at least 100,000, at least 10⁶, at least 10⁷, at least 10⁸ or at least 10⁹ or more members.

A “microfluidic device” is a device that is configured to control and manipulate fluids geometrically constrained to a small scale (e.g., sub-millimeter).

“Microtiter plate,” as used herein, refers to a plate with multiple “wells” diposed thereon in an rectangular array, where the wells may be used as small test tubes. Microtiter plates may, by way of non-limiting example, measure 128 mm×85 mm and may be made from a plastic such as polypropylene, polycarbonate, or polystyrene. A microplate may have 96 or 384 sample wells, may have 9600 wells, or more. The welles in some microtiter plates are arranged in a 2:3 rectangular matrix. Each microtiter plate well may contain a very small volume of liquid, ranging, for example, from about between tens of nanoliters to about a few milliliters.

A subject may be any human or non-human animal. A subject may be a person performing the instant method, a patient, a customer in a testing center, etc.

An “analyte,” as used herein is any substance that is suitable for testing in the present method. An analyte includes without limitation drugs, prodrugs, pharmaceutical agents, drug metabolites, biomarkers such as expressed proteins and cell markers, antibodies, antigens, proteins, hormones, polypeptides, glycoproteins, polysaccharides, lipids, viruses, cholesterol, polysaccharides, nucleic acids, genes, nucleic acids, and combinations thereof.

As used herein, a “biological sample” refers to any bodily byproduct, such as bodily fluids, that has been derived from a subject. The sample may be obtained directly from the subject in the form of liquid, or may be derived from the subject by first placing the bodily byproduct in a solution, such as a buffer. Exemplary samples include, but are not limited to, saliva, serum, blood, sputum, urine, sweat, lacrima, semen, feces, biopsies, mucus, etc.

A “biomarker,” as used herein, is any molecule or compound that is found in a sample of interest and that is known to be diagnostic of or associated with the presence of or a predisposition to a disease or condition of interest in the subject from which the sample is derived. Biomarkers include, but are not limited to, polypeptides or a complex thereof (e.g., antigen, antibody), nucleic acids (e.g., DNA, miRNA, mRNA), drug metabolites, lipids, carbohydrates, hormones, vitamins, etc., that are known to be associated with a disease or condition of interest.

The term “diagnostic,” as used herein, refers to the use of a method or an analyte for identifying, predicting the outcome of and/or predicting treatment response of a disease or condition of interest. A diagnosis may include predicting the likelihood of or a predisposition to having a disease or condition, estimating the severity of a disease or condition, determining the risk of progression in a disease or condition, assessing the clinical response to a treatment, and/or predicting the response to treatment.

A “processor,” “communication device,” “mobile device,” refer to computer systems that contain basic electronic elements (including one or more of a memory, input-output interface, central processing unit, instructions, network interface, power source, etc.) to perform computational tasks. The computer system may be a general purpose computer that contains instructions to perform a specific task, or may be a special-purpose computer.

A “site” or “location” refers to the local area in which a device or subject resides. A site may refer to a room within a building structure, such as a hospital, or a smaller geographically defined area within a larger geographically defined area. A remote site or remote location, with reference to a first site that is remote from a second site, is a first site that is physically separated from the second site by distance and/or by physical obstruction. The remote site may be a first site that is in a separate room from the second site in a building structure, a first site that is in a different building structure from the second site, a first site that is in a different city from the second site, etc.

As used herein, the term “sample collection site” refers to a location at which a sample may be obtained from a subject. A sample collection site may be, for example, a retailer location (e.g., a chain store, pharmacy, supermarket, or department store), a provider office, a physician's office, a hospital, the subject's home, a military site, an employer site, or other site or combination of sites. As used herein, the term “sample collection site” may also refer to a proprietor or representative of a business, service, or institution located at, or affiliated with, the site.

As used herein, “raw data” includes signals and direct read-outs from sensors, cameras, and other components and instruments which detect or measure properties or characteristics of a sample. For example, raw data includes voltage or current output from a sensor, detector, counter, camera, or other component or device; raw data includes digital or analog numerical output from a sensor, detector, counter, camera, or other component or device; and raw data may include digitized or filtered output from a sensor, detector, counter, camera, or other component or device. For example, raw data includes the output of a luminometer, which may include output in “relative light units” which are related to the number of photons detected by the luminometer. Raw data may include a JPEG, bitmap, or other image file produced by a camera. Raw data may include cell counts; light intensity (at a particular wavelength, or at or within a range of wavelengths); a rate of change of the output of a detector; a difference between similar measurements made at two times; a number of events detected; the number of events detected within a pre-set range or that meet a pre-set criterion; the minimum value measured within a time period, or within a field of view; the maximum value measured within a time period, or within a field of view; and other data. Where sufficient, raw data may be used without further processing or analysis. In other cases, raw data may be further processed or used for further analysis related to the sample, the subject, or for other purposes.

“Representative of a sample,” as used in reference to an output signal or raw data that are representative of the sample, refers to the output signal or raw data reflecting a measured property of the sample or a portion thereof, e.g., reflecting the amount of analyte of interest present in the sample. For instance, the intensity of a fluorescence signal representative of a sample may be more intense in a fluorescently labeled sample that contains more analyte of interest than the intensity of a fluorescence signal representative of a fluorescently labeled sample that contains less analyte.

As used herein, “Clinical Laboratory Improvement Amendments” and “CLIA” refer to sections of 42 U.S.C. Part F, e.g., subpart 2, sections 263a through 263a7, Federal Regulations 42 CFR Chapter W (sections 493.1 to 493.2001), and related laws, regulations, and as amended. Regulations pursuant to CLIA are administered by the Centers for Medicare and Medicaid Services (CMS) of the United States Department of Health and Human Services.

“Process management,” as used herein, refers to any number of methods and systems for planning and/or monitoring the performance of a process, such as a sample analysis process.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

### DETAILED DESCRIPTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the present disclosure.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

It must be noted that as used herein and in the appended claims, the singular forms “a,” “an,” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

One with skill in the art will appreciate that the present invention is not limited in its application to the details of construction, the arrangements of components, category selections, weightings, pre-determined signal limits, or the steps set forth in the description or drawings herein. The invention is capable of other embodiments and of being practiced or being carried out in many different ways.

The practice of various embodiments of the present disclosure employs, unless otherwise indicated, conventional techniques of immunology, biochemistry, chemistry, molecular biology, microbiology, cell biology, genomics and recombinant DNA, which are within the skill of the art. See Green and Sambrook, MOLECULAR CLONING: A LABORATORY MANUAL, 4^{th} edition (2012); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

### Methods

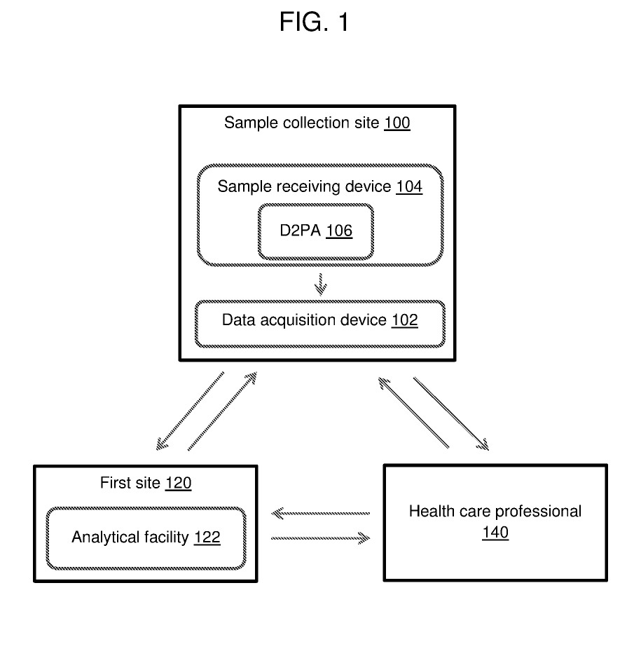

As summarized above, aspects of the present disclosure include a method for analyzing a biological sample collected from a subject. Certain embodiments of the present disclosure are described with reference to the drawings. In implementing the present method, data transmitted from a data acquisition device 102 is received at a first site 120 (FIG. 1). The data transmitted by the data acquisition device may be a readout from a disc-coupled dots-on-pillar array (D2PA) device 104 that produces an output signal when a biological sample is applied to the D2PA device. In some instances, the biological sample is collected from a subject at a second site 100, which may be a point-of-care location or a sample collection site, such as a doctor's office, an emergency room, or a home. A D2PA device may be any suitable device that contains a D2PA 106 that is configured to bind and detect an analyte of interest. In some embodiments, the D2PA device 104 is a microfluidic device or a microtiter plate that contains one or more D2PAs. The D2PA device may produce an output signal, e.g., a fluorescence readout, which indicates the presence and/or amount of the analyte in the sample. The output signal may be read by a data acquisition device 102 which is configured to read the output signal from the D2PA device 104. In some embodiments, the data acquisition device 102 is a mobile device, such as a smart phone, that includes a camera to capture an image of the D2PA device 104 and the output signal, and a network communication interface, such as a wireless interface, to transmit the data containing raw data representative of the output signal to a first site 120 for analysis. The first site 120 may be the same site as the sample collection site 100, e.g., in the same room, or may be a remote site from the sample collection site 100, e.g., in a different room in the same building, in a different building, in a different city, etc. The first site 120 may be an analytical facility, e.g., a laboratory, including an authorized analytical facility. At the first site 120, the data may be analyzed to generate an evaluation of the data. In some embodiments, one or more process management operations may be performed to provide oversight over the analysis of the sample such that the evaluation may be used to screen, diagnose or treat a subject. In some embodiments, a health care professional 140 performs at least part of the analysis to generate the evaluation. In some cases, the health care professional 140 may be at the first site 120 or, if the first site is a remote site, at the sample collection site 100. In some embodiments, the one or more process management operations may be performed at the sample acquisition site 100. In some instances, the evaluation may be generated without involvement of any health care professional in the analysis of the data, for example, where the data is analyzed by a software.

A sample collection site 100 may be a point of service (POS) location. Any disclosure herein of a sample collection site 100 may also apply to a point of service location and vice versa. A point of service location where a sample may be collected from a subject or provided by a subject may be a location remote to the analytical facility 122, e.g., a laboratory. The sample collection site may have a separate facility from a laboratory. The sample may or may not be collected fresh from the subject at the sample collection site. Alternatively, the sample may be collected from the subject elsewhere and brought to the sample collection site. A sample collection site at a point of service location may be a blood collection center, or any other bodily fluid collection center. The sample collection site may be a biological sample collection center. In some embodiments, a sample collection site may be a retailer. Other examples of sample collection sites may include hospitals, clinics, health care professionals' offices, schools, day-care centers, health centers, assisted living residences, government offices, traveling medical care units, mobile units, emergency vehicles (e.g., air, boat, ambulance), or the home. For example, a sample collection site may be a subject's home. A sample collection site may be at a sample acquisition site and/or health assessment and/or treatment locations (which may include any of the sample collection sites described elsewhere herein including but not limited to emergency rooms, doctors' offices, urgent care, tents for screening (which may be in remote locations), a health care professional walking into someone's house to provide home care). A sample collection site may be any location where a sample from the subject is received by the D2PA device. Any location may be designated as a sample collection site. The designation may be made by any party, including but not limited to the laboratory, entity associated with the laboratory, governmental agency, or regulatory body. Any description herein relating to sample collection site or point of service may relate to or be applied to retailers, hospitals, clinics, or any other examples provided herein and vice versa.

A D2PA device and/or data acquisition device may be provided at the sample collection site. The D2PA device may be configured to accept a sample. The D2PA device may accept a sample collected from a subject at the sample collection site, or that the subject or subject's proxy brings to the service location. The D2PA device may directly collect the sample from the subject, or an intermediate device or technique may be used to collect the sample from the subject.

An analytical facility 122 can be an entity or facility, e.g., a laboratory, or system or device capable of performing a clinical test or analyzing collected data. A laboratory can provide controlled conditions in which scientific research, experiments, and measurement can be performed. The laboratory can be a medical laboratory or clinical laboratory where tests can be done on clinical specimens, or analysis can occur on data collected from clinical specimens, in order to get information about the health of a patient as pertaining to the screening, diagnosis, prognosis, treatment, and/or prevention of disease. A clinical specimen may be a sample collected from a subject. A clinical specimen may be collected from the subject at a sample collection site that is at a separate facility from the laboratory, as described in further detail elsewhere herein. The clinical specimen may be collected from the subject using a device, e.g., a D2PA device, which is placed at a designated sample collection site.

In some embodiments, a laboratory may be a certified laboratory. The certified laboratory may be an authorized analytical facility. In some embodiments, authorized analytical facilities may include contracted analytical facilities. For example, a certified laboratory or other laboratory may send images to experts at another laboratory (which may be a certified laboratory) for analysis.

Any description herein of a laboratory may apply to an authorized analytical facility and vice versa. In some instances, the laboratory may be certified by a governmental agency or professional association. A laboratory may receive certification or oversight by a regulatory body. In one example, the laboratory may be certified by an entity, such as Centers for Medicare & Medicaid Services (CMS), College of American Pathologists, ISO standards 15189 or 17025 or equivalents thereof. For instance, an authorized analytical facility may be a Clinical Laboratory Improvement Amendments (CLIA) certified laboratory in the United States or its equivalent in a foreign jurisdiction.

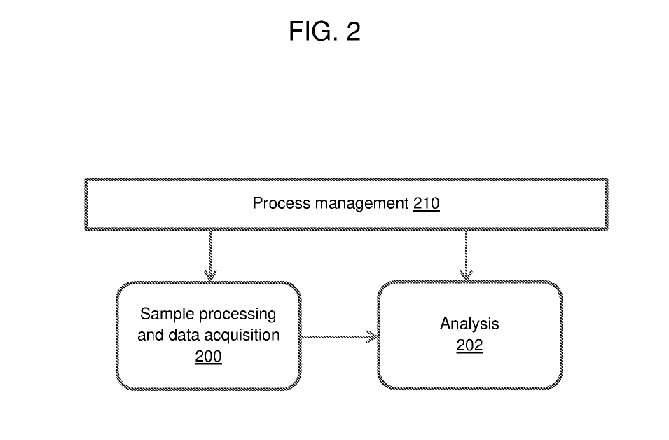

Further aspects of the present disclosure includes providing a process management operation 210 (FIG. 2), including quality assurance of the sample handling and data acquisition 200 as well as oversight of the data analysis 202, such that the evaluation for the biological sample can be used for screening, diagnosis or treatment of the subject. The process management operation may include, but is not limited to, quality assurance of the raw data, quality assurance of the data acquisition device and/or D2PA device, oversight of the use of the data acquisition device and/or D2PA device, oversight of the analysis performed on the data to generate the evaluation, and authorization of access to the evaluation and/or the data comprising the raw data by the health care professional.

An analytical facility 122, e.g., a laboratory, may be in communication with a sample collection site 100 and a health care professional 140. The laboratory may be in communication with any number of sample collection sites and health care professionals. For example, the laboratory may be in communication with one or more, two or more, three or more, five or more, ten or more, fifteen or more, twenty or more, 30 or more, 50 or more, 100 or more, 200 or more, 500 or more, 1000 or more, 5000 or more, 10,000 or more, 100,000 or more, or 1,000,000 or more sample collection sites and/or health care professionals. In some systems, one, two, three, four, or more laboratories may be provided that may communicate with any number of sample collection sites and/or health care professionals. The laboratories may or may not communicate with one another. The sample collection sites, laboratories, and/or health care professionals may be scattered geographically at any location. In some embodiments, the sample collection sites and/or health care professionals in communication with a laboratory may be in the same geographic region (e.g., town, city, state, region, country). Alternatively, the sample collection sites and/or health care professionals in communication with a laboratory may be scattered anywhere globally.

The analytical facility 122, e.g., laboratory, may communicate with the health care professional 140 and the sample collection site in any manner known in the art. In some embodiments, the laboratory may communicate directly with a data acquisition device located at the sample collection site. Such communications may be via electronic signals, radiofrequency signals, optical signals, cellular signals, or any other type of signals that may be transmitted via a wired or wireless connection.

Any transmission of data or description of electronic data or transmission described elsewhere herein may occur via electronic signals, radiofrequency signals, optical signals, cellular signals, or any other type of signals that may be transmitted via a wired or wireless connection. For example, data may be transmitted electronically from a sample collection site to an analytical facility, e.g., a laboratory, and vice versa. Data may be transmitted from a data acquisition device which may be at the sample collection site to the laboratory and vice versa. Similarly, data may be transmitted electronically from a laboratory to a health care professional 140 and vice versa. The communications may be over a network such as a local area network (LAN), wide area network (WAN) such as the Internet, personal area network, a telecommunications network such as a telephone network, cell phone network, mobile network, a wireless network, a data-providing network, or any other type of network. The communications may utilize wireless technology, such as Bluetooth or RTM technology. Alternatively, various communication methods may be utilized, such as a dial-up wired connection with a modem, a direct link such as TI, ISDN, or cable line. In some embodiments, a wireless connection may be using exemplary wireless networks such as cellular, satellite, or pager networks, GPRS, or a local data transport system such as Ethernet or token ring over a LAN. In some embodiments, the data acquisition device may communicate wirelessly using infrared communication components. A data acquisition device, personal computer, server, laptop computer, tablet, mobile phone, cell phone, satellite phone, smartphone (e.g., iPhone®, Android®, Blackberry®, Palm®, Symbian®, Windows®), personal digital assistant, Bluetooth® device, pager, land-line phone, or other network device may be used in order to provide communications. Such devices may be communication-enabled devices.

A health care professional may include a person or entity that is associated with the health care system. A health care professional may be a medical health care provider. A health care professional may be a doctor. A health care professional may be an individual or an institution that provides preventive, curative, promotional or rehabilitative health care services in a systematic way to individuals, families and/or communities. Examples of health care professionals may include physicians (including general practitioners and specialists), dentists, audiologists, speech pathologists, physician assistants, nurses, midwives, pharmaconomists/pharmacists, dietitians, therapists, psychologists, chiropractors, clinical officers, physical therapists, phlebotomists, occupational therapists, optometrists, emergency medical technicians, paramedics, medical laboratory technicians, medical prosthetic technicians, radiographers, social workers, and a wide variety of other human resources trained to provide some type of health care service. A health care professional may or may not be certified to write prescriptions. A health care professional may work in or be affiliated with hospitals, health care centers and other service delivery points, or also in academic training, research and administration. Some health care professionals may provide care and treatment services for patients in private homes. Community health workers may work outside of formal health care institutions. Managers of health care services, medical records and health information technicians and other support workers may also be health care professionals or affiliated with a health care provider.

In some embodiments, the health care professional may already be familiar with the subject or have communicated with the subject. The subject may be a patient of the health care professional. In some instances, the health care professional may have prescribed the subject to undergo a clinical test. The health care professional may have instructed or suggested to the subject to undergo a clinical test conducted at the sample collection site or by the laboratory. In one example, the health care professional may be the subject's primary care physician. The health care professional may be any type of physician for the subject (including general practitioners, and specialists).

A health care professional 140 may receive a report from an authorized analytical facility. The health care professional that receives a report may be an ordering health care professional or health care professional in the analytical facility and/or sample collection site.

Further aspects of the subject method are described in detail below.

### Disk-Coupled Dots-on-Pillar Antenna Array (D2PA) and D2PA Device

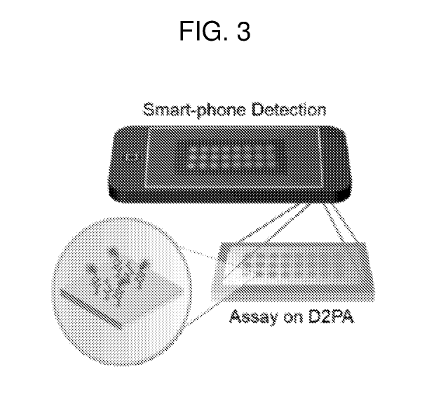

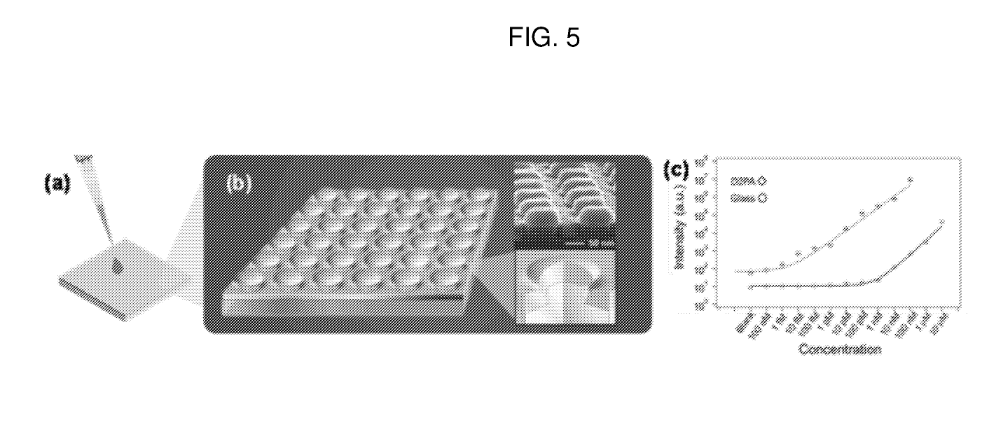

Aspects of the present disclosure include a disk-coupled dots-on-pillar antenna array (D2PA) 106 and a D2PA device 104. The terms “disk-coupled dots-on-pillar antenna array” and “D2PA” as used herein refer to an array that includes: (a) substrate; and (b) a D2PA structure, on the surface of the substrate, containing one or a plurality of pillars extending from a surface of the substrate, wherein at least one of the pillars includes a pillar body, metallic disc on top of the pillar, metallic back plane at the foot of the pillar, the metallic back plane covering a substantial portion of the substrate surface near the foot of the pillar; metallic dot structure disposed on sidewall of the pillar. FIGS. 3 and 5 illustrate exemplary D2PA structures. The D2PA amplifies a light signal that is proximal to the surface of the D2PA. The D2PA enhances local electric field and local electric field gradient in regions that is proximal to the surface of the D2PA. The light signal includes light scattering, light diffraction, light absorption, nonlinear light generation and absorption, Raman scattering, chromaticity, luminescence that includes fluorescence, electroluminescence, chemiluminescence, and electrochemiluminescence.

Details of the physical structure of disk-coupled dots-on-pillar antenna arrays, methods for their fabrication, methods for linking capture agents to disk-coupled dots-on-pillar antenna arrays and methods of using disk-coupled dots-on-pillar antenna arrays to detect analytes are described in a variety of publications including WO2012024006, WO2013154770, Li et al (Optics Express 2011 19, 3925-3936), Zhang et al (Nanotechnology 2012 23: 225-301); and Zhou et al (Anal. Chem. 2012 84: 4489) which are incorporated by reference for those disclosures.

The D2PA may contain a capture agent that binds to an analyte of interest in a sample.

The capture agent may vary depending on the analyte of interest to be detected in a sample. In some cases, the capture agent is an antibody, an antibody epitope, a nucleic acid binding protein, a nucleic acid, etc., as discussed above. In some embodiments, the capture agent is stably bound to the exterior surface of the D2PA molecular adhesion layer by reacting with a capture-agent-reactive group, i.e., a reactive group that can chemically react with capture agents, e.g., an amine-reactive group, a thiol-reactive group, a hydroxyl-reactive group, an imidazolyl-reactive group and a guanidinyl-reactive group, etc. In one embodiment, capture agent can be nucleic acid to capture proteins, or capture agent can be proteins that capture nucleic acid, e.g., DNA, RNA. Nucleic acid can bind to proteins through sequence-specific (tight) or non-sequence specific (loose) bond.

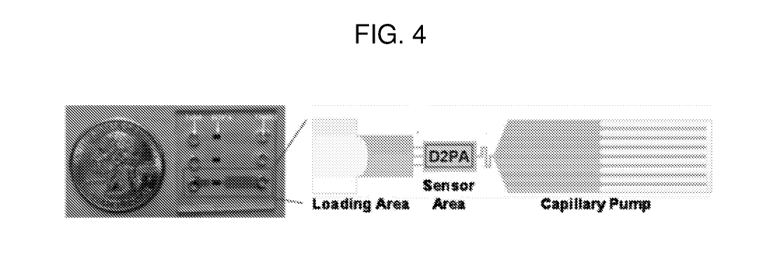

In certain embodiments, the D2PA 106 is integrated into a solid support or platform to form a D2PA device 104. In some embodiments, the D2PA is integrated into a nanosensor device that includes a platform or support. In certain embodiments, the nanosensor device is a microfluidic platform or device (FIG. 4). The microfluidic device may be configured to have different areas for receiving a sample, detecting analytes in the sample with a D2PA, collecting waste material in a reservoir, etc. Thus, in certain embodiments, the microfluidic channel platform may include fluid handling components to direct a sample applied to a sample receiving area of the microfluidic device to a D2PA configured to detect an analyte, as described above. The fluid handling components may be configured to direct one or more fluids through the microfluidic device. In some instances, the fluid handling components are configured to direct fluids, such as, but not limited to, a sample solution, buffers and the like. Liquid handling components may include, but are not limited to, passive pumps and microfluidic channels. In some cases, the passive pumps are configured for capillary action-driven microfluidic handling and routing of fluids through the microfluidic device disclosed herein. In certain instances, the microfluidic fluid handling components are configured to deliver small volumes of fluid, such as 1 mL or less, such as 500 μL or less, including 100 μL or less, for example 50 μL or less, or 25 μL or less, or 10 μL or less, or 5 μL or less, or 1 μL or less. Thus, in certain embodiments, no external source of power is required to operate the microfluidic device and perform the present method.

In certain embodiments, the microfluidic device has dimensions in the range of 5 mm×5 mm to 100 mm×100 mm, including dimensions of 50 mm×50 mm or less, for instance 25 mm×25 mm or less, or 10 mm×10 mm or less. In certain embodiments, the microfluidic device has a thickness in the range of 5 mm to 0.1 mm, such as 3 mm to 0.2 mm, including 2 mm to 0.3 mm, or 1 mm to 0.4 mm.

In some embodiments, the D2PA device 104 is a disposable system.

In certain embodiments, the D2PA 106 is integrated on a dipstick structure or a lateral flow format, examples of which is described in, e.g., U.S. Pat. No. 6,660,534, incorporated herein by reference.

In certain embodiments, the D2PA 106 is disposed within a container, e.g., a well of a multi-well plate. The D2PA also can be integrated into the bottom or the wall of a well of a multi-well plate.

In some embodiments, a support containing a D2PA 106, such as a microfluidic device or multi-well plate, may have an identifier for the D2PA that is contained in the support. An identifier may be a physical object formed on the support, such as a microfluidic device. For example, the identifier may be read by a data acquisition device, e.g., a mobile device such as a mobile phone or a smart phone. In some embodiments, a camera may capture an image of the identifier and the image may be analyzed to identify the D2PA contained in the microfluidic device. In one example, the identifier may be a barcode. A barcode may be a 1D or 2D barcode. In some embodiments, the identifier may emit one or more signal that may identify the signal enhancing detector. For example, the identifier may provide an infrared, ultrasonic, optical, audio, electrical, or other signal that may indicate the identity of the D2PA. The identifier may utilize a radiofrequency identification (RFID) tag.

The identifier may contain information that allows determination of the specific type of D2PA present in a microfluidic device or multi-well plate. In certain embodiments, the identifier provides a key to a database that associates each identifier key to information specific to the type of D2PA present in a microfluidic device or multi-well plate. The information specific to the type of D2PA may include, but are not limited to, the identity of the analytes which the D2PA configured to detect, the coordinates of the position where a specific analyte may bind on the D2PA, the sensitivity of detection for each analyte, etc. The database may contain other information relevant to a specific D2PA, including an expiration date, lot number, etc. The database may be present on a mobile device, provided on a computer-readable medium, or may be on a server accessible by a mobile device.

The D2PA device 104 may be configured to receive a biological sample collected from the subject. The D2PA device may be configured to receive any suitable volume of sample. Examples of sample volumes may include, but are not limited to, about 10 mL or less, 5 mL or less, 3 mL or less, 1 microliter (μL, also “uL” herein) or less, 500 μL, or less, 300 μL, or less, 250 μL, or less, 200 μL, or less, 170 μL, or less, 150 μL, or less, 125 μL, or less, 100 μL, or less, 75 μL, or less, 50 μL, or less, 25 μL, or less, 20 μL, or less, 15 μL, or less, 10 μL, or less, 5 μL, or less, 3 μL, or less, 1 μL, or less. The amount of sample may be about a drop of a sample. The amount of sample may be the amount collected from a pricked finger or fingerstick. The amount of sample may be the amount collected from a microneedle or a venous draw.

The D2PA device 104 may be configured to receive a sample without further processing after the sample has been obtained from the source, or may receive a sample that has been processed, e.g., to enrich for an analyte of interest, remove large particulate matter, dissolve or resuspend a solid sample, etc.

The method may include receiving a sample collected at one time, or at a plurality of times. Samples collected over time may be aggregated and/or processed (by applying to a D2PA and obtaining a measurement of the amount of analyte in the sample) individually. In some instances, measurements obtained over time may be aggregated and may be useful for longitudinal analysis over time to facilitate screening, diagnosis, treatment, and/or disease prevention.

A implementation of a method of using a D2PA device 104 containing a D2PA 106 for detecting an analyte in a sample may be described as follows. The D2PA may include a capture agent that specifically binds to an analyte of interest. Binding of the analyte to the capture agent results in an analyte-capture agent complex that is immobilized on the D2PA. Once the capture agent binds to the analyte of interest to form a detectably labeled, analyte-capture agent complex, the amount of bound analyte may be measured by reading the D2PA. Thus, the amount of analyte in the sample may be inferred from the amount of labeled analyte measured from the D2PA.

An analyte in the sample that is captured by the D2PA may be labeled with a detectable label that binds, directly or indirectly, to the captured analyte. An analyte in the sample may be labeled using any convenient method, and in some cases is labeled before applying the sample to the D2PA and binding the labeled analyte to the capture agent, or is labeled after, or at the same time as binding of the analyte to the capture agent on the D2PA. The D2PA may be washed as necessary, for example, to remove any unbound sample components, e.g, proteins, nucleic acids, compounds, etc., that are not of interest, or to remove unbound label, etc.

In certain embodiments, the D2PA is configured to enhance the signal from a detectable label that is proximal to the surface of the D2PA by 10³ fold or more, for example, 10⁴ fold or more, 10⁵ fold or more, 10⁶ fold or more, 10⁷ fold or more, including 10⁸ fold or more, where the signal may be enhanced by a range of 10³ to 10⁹ fold, for example, 10⁴ to 10⁸ fold, or 10⁵ to 10⁷ fold, compared to a detectable label that is not proximal to the surface of the D2PA, i.e., compared to a detectable label bound to an analyte on a conventional ELISA plate, on a conventional nucleic acid microarray, suspended in solution, etc. In certain embodiments, the D2PA is configured to enhance the signal from a detectable label that is proximal to the surface of the D2PA by 10³ fold or more, for example, 10⁴ fold or more, 10⁵ fold or more, 10⁶ fold or more, 10⁷ fold or more, including 10⁸ fold or more, where the signal may be enhanced by a range of 10³ to 10⁹ fold, for example, 10⁴ to 10⁸ fold, or 10⁵ to 10⁷ fold, compared to an analyte detecting array that is not configured to enhance the signal using a physical amplification process, as described above. In certain embodiments, the D2PA is configured to have a detection sensitivity of 0.1 nM or less, such as 10 pM or less, or 1 pM or less, or 100 fM or less, such as 10 fM or less, including 1 fM or less, or 0.5 fM or less, or 100 aM or less, or 50 aM or less, or 20 aM or less. In certain embodiments, the D2PA is configured to have a detection sensitivity in the range of 10 aM to 0.1 nM, such as 20 aM to 10 pM, 50 aM to 1 pM, including 100 aM to 100 fM. In some instances, the D2PA is configured to be able to detect analytes at a concentration of 1 ng/mL or less, such as 100 pg/mL or less, including 10 pg/mL or less, 1 pg/mL or less, 100 fg/mL or less, 10 fg/mL or less, or 5 fg/mL or less. In some instances, the D2PA is configured to be able to detect analytes at a concentration in the range of 1 fg/mL to 1 ng/mL, such as 5 fg/mL to 100 pg/mL, including 10 fg/mL to 10 pg/mL. In certain embodiments, the D2PA is configured to have a dynamic range of 5 orders of magnitude or more, such as 6 orders of magnitude or more, including 7 orders of magnitude or more.

In certain instances, the period of time from applying the sample to the D2PA device 104 to reading the D2PA device may range from 1 second to 30 minutes, such as 10 seconds to 20 minutes, 30 seconds to 10 minutes, including 1 minute to 5 minutes. In some instances, the period of time from applying the sample to the signal enhancing detector to generating an output that can be received by the device may be 1 hour or less, 30 minutes or less, 15 minutes or less, 10 minutes or less, 5 minutes or less, 3 minutes or less, 1 minute or less, 50 seconds or less, 40 seconds or less, 30 seconds or less, 20 seconds or less, 10 seconds or less, 5 seconds or less, 2 seconds or less, 1 second or less, or even shorter. In some instances, the period of time from applying the sample to the signal enhancing detector to generating an output that can be received by the device may be 100 milliseconds or more, including 200 milliseconds or more, such as 500 milliseconds or more, 1 second or more, 10 seconds or more, 30 seconds or more, 1 minute or more, 5 minutes or more, or longer.

### Data Acquisition Device

Further aspects of the present disclosure includes a data acquisition device 102 configured to read an output signal from a D2PA device 104 to generate data containing raw data that is representative of the biological sample and transmit the data to the first site 120. The data acquisition device may be any suitable device that is configured to read a D2PA device and transmit the raw data to the first site. In some cases, the data acquisition device is an instrument specialized to receive the D2PA device and read an output signal from the D2PA device. The data acquisition device may include an optical recording apparatus, a holder for receiving a D2PA device and a box of D2PA devices. The optical recording apparatus may include, for example, a camera, a display, electronic memory and a keypad for manual data entry. The optical data acquisition device may communicate with a communication device at the first site using, for example, a wireless connection, an optical link, a network connection or any other suitable form of communication link that uses any form of communication protocol to transfer information.

In some embodiments, the data acquisition device 102 includes an optical recording apparatus that is configured to acquire a light signal from the D2PA, e.g., acquire an image of the D2PA (FIG. 3). In certain instances, the optical recording apparatus is a camera, such as a digital camera. The term “digital camera” denotes any camera that includes as its main component an image-taking apparatus provided with an image-taking lens system for forming an optical image, an image sensor for converting the optical image into an electrical signal, and other components, examples of such cameras including digital still cameras, digital movie cameras, and Web cameras (i.e., cameras that are connected, either publicly or privately, to an apparatus connected to a network to permit exchange of images, including both those connected directly to a network and those connected to a network by way of an apparatus, such as a personal computer, having an information processing capability). In one example, reading the D2PA may include video imaging that may capture changes over time. For example, a video may be acquired to provide evaluation on dynamic changes in the sample applied to the D2PA.

In certain embodiments, the optical recording apparatus has a sensitivity that is lower than the sensitivity of a high-sensitivity optical recording apparatus used in research/clinical laboratory settings. In certain cases, the optical recording apparatus used in the subject method has a sensitivity that is lower by 10 times or more, such as 100 times or more, including 200 times or more, 500 times or more, or 1,000 times or more than the sensitivity of a high-sensitivity optical recording apparatus used in research/clinical laboratory settings.

The data acquisition device 102 may include a microprocessor (e.g., any type of processor). The data acquisition device may also include any type of computer memory or any other type of electronic storage medium that is located either internally or externally to the data acquisition device, such as, for example, a random access memory (RAM). According to exemplary embodiments, the RAM may contain, for example, the operating program for the data acquisition device. As will be appreciated based on the following description, the RAM can, for example, be programmed using conventional techniques known to those having ordinary skill in the art of computer programming. The actual source code or object code for carrying out the steps of, for example, a computer program can be stored in the RAM.

The data acquisition device 102 may include a communications port (e.g., any type of communications port through which electronic information can be communicated over a communications connection, whether locally or remotely) with which the data acquisition device can communicate with a communication device, e.g., a server. The data acquisition device may also include the holder that, for example, allows insertion of the D2PA device. The data acquisition device may also include a user interface. The user interface may be any type of computer monitor or display device on which graphical and/or textual information can be displayed to a user (e.g., through a graphical user interface) and which allows a user to enter information (e.g., commands and the like) through, for example, a keyboard, a touch-screen, any type of pointing device, electronic pen, and the like. For example, the user interface can be configured to receive instructions from the operator of the data acquisition device. Each department within a hospital may have one or more data acquisition devices.

In certain embodiments, the data acquisition device 102 may have a video display. Video displays may include components upon which a display page may be displayed in a manner perceptible to a user, such as, for example, a computer monitor, cathode ray tube, liquid crystal display, light emitting diode display, touchpad or touchscreen display, and/or other means known in the art for emitting a visually perceptible output. In certain embodiments, the data acquisition device is equipped with a touch screen for displaying information, such as the image acquired from the detector and/or a report generated from the processed data, and allowing information to be entered by the subject.

In some embodiments, the data acquisition device 102 is a mobile device, such as a mobile phone or a smart phone. Any suitable mobile device configured to read the D2PA may be used in the present method. Devices configured to read the D2PA are described in, e.g., U.S. Provisional Application Ser. No. 62/066,777, filed on Oct. 21, 2014, which is incorporated herein by reference. In some embodiments, the mobile device may contain instructions, e.g., a medical app, in a memory location, wherein the instructions, when executed, provides a user interface to control the data acquisition device such that a signal output from the D2PA device may be read by the data acquisition device to generate data containing raw data.

Any suitable method may be used to read the D2PA device 104 to obtain a measurement of the amount of analyte in the sample. In some embodiments, reading the D2PA device includes obtaining an electromagnetic signal from the detectable label bound to the analyte in the D2PA device. In certain embodiments the electromagnetic signal is a light signal. The light signal obtained may include the intensity of light, the wavelength of light, the location of the source of light, and the like. In particular embodiments, the light signal produced by the label has a wavelength that is in the range of 300 nm to 900 nm. In certain embodiments, the light signal is read in the form of a visual image of the D2PA device.

In certain embodiments, reading the D2PA device 104 includes providing a source of electromagnetic radiation, e.g., light source, as an excitation source for the detectable label bound to the biomarker in the D2PA device. The light source may be any suitable light source to excite the detectable label. Exemplary light sources include, but are not limited to, sun light, ambient light, UV lamps, fluorescent lamps, light-emitting diodes (LEDs), photodiodes, incandescent lamps, halogen lamps, and the like.

The data provided by the data acquisition device 102 may include data relating to a sample from a subject. The data may be information necessary and/or sufficient for a qualitative and/or quantitative evaluation of the sample. The data may include information for oversight. The data may include information for analysis. The data may be an electronic representation of a sample. An electronic representation of a sample may include an electronic representation of the entire sample and/or any portion thereof. The data may be electronic data. In some instances, the data may be electronic bits representative of the sample or reaction or reagents. The data may be digital and/or analog. The data may be representative of one or more measurable parameter relating to, based on, or of the sample.

The raw data may be representative of a sample and/or any portion thereof. In some embodiments, the data is representative of a preparation of the collected biological sample. The data may be collected prior to, during, and/or after the preparation of the sample. The data may be collected over time. The data may comprise information of one or more conditions under which a preparation of the collected biological sample occurs. Examples of such conditions may comprise one or more characteristics listed from the group: amount of the biological sample, concentration of the biological sample, quality of the biological sample, temperature, or humidity. Such conditions may include environmental conditions. Environmental conditions may refer to conditions of the sample, and/or the surroundings of the sample. The environmental conditions may be provided prior to, during, and/or after the sample is received by the D2PA device and/or data is transmitted by the data acquisition device.

The raw data may include amounts, concentrations, proportions, purity, or other information of sample, reagents, diluents, wash, dyes or any other material that may be involved in the preparation of a sample, reactions, and/or controls/calibrations on the data acquisition and/or D2PA devices. Physical and/or chemical properties of a sample and/or other materials, and/or a chemical reaction may be measured at one or more points in time, and may be aggregated as data. In some embodiments, the data may determine whether a sample, reagent, diluents, wash, dye, or any other material is suitable for use in the D2PA device for said sample preparation and/or to permit subsequent qualitative and/or quantitative evaluation. For example, the data may be indicative of any error conditions that may indicate a sample and/or any of the other materials have gone bad, or are otherwise unsuitable. In some instances, data is collected during any processes the data acquisition and/or D2PA devices are performing.

In some embodiments, the raw data may be representative of a chemical reaction which may be run by the D2PA device. The chemical reaction may include a chemical reaction with the sample, or without the sample. The chemical reaction may include one or more reagents that may react with the sample. The chemical reaction may include a control or calibration reaction. The data representative of the reaction may include one or more measurement of the chemical reaction. The data may also include the rate or speed of the chemical reaction, and/or the acceleration of the chemical reaction. The data may include how complete a chemical reaction is (e.g., whether the chemical reaction has started, whether the chemical reaction is taking place, whether the chemical reaction is complete, how far along the chemical reaction is—e.g., 10%, 50%, etc.). The data may comprise information about a control reaction and a chemical reaction involving the biological sample. These reactions may occur simultaneously and/or sequentially. The data may pertain to one or more chemical reactions that may or may not occur simultaneously. The data may pertain to one or more sample preparation step that may or may not occur simultaneously. The data may also include physical processing, such as centrifugation, pulveration, or any other actions described herein, which may be represented through bits of data. The data can be utilized for oversight functionally performed on-board, remotely by a health care professional 140, and/or an external device configured to render such oversight.

In some examples, the raw data may be one or more image, and/or audio data representative of the sample. An image may be a digital image or an analog image. The audio data may be digital and/or analog. The data may include a video representative of the sample. An image may include a video image. The data may include electronic data representative of a digital image and/or audio data of the sample. In one example, the data may include video imaging that may capture changes over time. For example, a video may be provided to provide evaluation on dynamic actions, such as lysing, agglutination, mixing, movement of cells or other molecules in a sample or matrix, or assays.

The data may be collected at one time, or at a plurality of times. The data may be collected at discrete points in time, or may be continuously collected over time. Data collected over time may be aggregated and/or analyzed. In some instances, data may be aggregated and may be useful for longitudinal analysis over time to facilitate screening, diagnosis, treatment, and/or disease prevention.

Raw data may be collected from data acquisition and D2PA devices 102, 104 over time. The aggregated data from a single data acquisition device and a single D2PA device for a given sample may be useful to facilitate the qualitative and/or quantitative evaluation of the sample. For example, it may be useful to determine how a sample reacts and/or changes over time in order to provide a screening, diagnosis, treatment, and/or disease prevention.

In some embodiments, data containing raw data may be displayed in a lab report, medical record, or any other type of display. The display may show patient health, provider's level of care, disease regression, progression, and/or onset through longitudinal analysis of high integrity data that is may be obtainable more frequently or obtained frequently through the described infrastructure over time.

Data containing raw data may be collected from multiple data acquisition and/or D2PA devices. The aggregated data from multiple data acquisition and/or D2PA devices may be useful to facilitate the qualitative and/or quantitative evaluation of the sample. The aggregated data may include data relating to samples collected from a single subject, received at the multiple data acquisition and/or D2PA devices. Alternatively, the aggregated data may include data relating to samples collected from other subjects, received at the multiple data acquisition and/or D2PA devices. The aggregated data may be collected and/or stored in a database. The database may be accessed to provide data to perform a longitudinal analysis that takes past collected data into account. Trends, and changes over time may be monitored. The multiple devices may be standardized and/or may provide data that is of sufficient quality, precision, and/or accuracy in order to aggregate the data and perform a longitudinal analysis therefrom. Very little or no variation may be provided between data acquisition and/or D2PA devices. The data acquisition and/or D2PA devices may also create standardized environments in which the sample preparation may occur. The standardized environments may also be provided during a chemical reaction. The data acquisition and/or D2PA devices may also provide standardized pre-analytic steps. The multiple data acquisition and/or D2PA devices may be distributed globally. This may provide a global evaluation infrastructure, which may better permit the monitoring of disease progression and/or regression. By standardizing data acquisition and/or D2PA devices, data may be longitudinally analyzed looking at velocity of markers in one or more subject over time. The data may be analyzed and/or displayed in a form of a lab report or electronic medical record or decision support system for consumers, providers, and/or payers (e.g., health plans, employers, governmental payers, etc.). Such display may include displays of data over time, which may include trending analysis or other analysis relating to changes in values, rates of changes, or rates of rates of change.

The raw data may be of a quality suitable for a longitudinal analysis over time. The suitable quality of data may be useful for lab reports and/or electronic medical records that may incorporate data collected over time. This may include data collected over long periods of time (e.g., multiple visits, or based off multiple samples), or shorter periods of time (within a single visit, or based on single received sample). The data may have a sufficient quality, precision, and/or accuracy for longitudinal analysis. For example, the sample may be collected from a subject a plurality of times. The sample may be collected from the subject at different times. The samples may be collected at predetermined intervals or according to a predetermined schedule. Alternatively, samples may be collected from the subject when one or more condition or event triggers the collection. Multiple collections of samples may permit the sample to be analyzed over a period of time, thereby permitting longitudinal analysis. In some embodiments, in order to permit longitudinal analysis, the data may have a high degree of precision and/or accuracy. In one example, the data may have a coefficient of variation of 20% or less, 15% or less, 10% or less, 9% or less, 8% or less, 7% or less, 6% or less, 5% or less, 4% or less, 3% or less, 2% or less, 1% or less, 0.5% or less, or 0.1% or less over time. In some instances, the multiple devices may provide data having a coefficient of variation of 20% or less, 15% or less, 10% or less, 9% or less, 8% or less, 7% or less, 6% or less, 5% or less, 4% or less, 3% or less, 2% or less, 1% or less, 0.5% or less, or 0.1% or less over time.

The raw data over time may be analyzed longitudinally. This may include the change in data over time, the rate of change of data over time, the rate of change of the rate of change of data over time, or any derivative thereof. For example, velocity and/or acceleration of data change may be collected and/or analyzed. The increase and/or decrease in the data values and/or the various rates of change may be beneficial in determining a screening, diagnosis, treatment, and/or disease prevention.

In embodiments, a data acquisition device may be configured to control the temperature within the device, or within a portion of the data acquisition device. Such control improves the reproducibility of measurements made within the device, may unify or provide regularity of conditions for all samples, and reduce the variability of measurements and data, e.g., as measured by the coefficient of variance of multiple measurements or replicate measurements. Temperature information may be useful for quality control. In embodiments, a data acquisition device may monitor temperature and control its internal temperature. Temperature control may be useful for quality control. A data acquisition device that monitors and controls its temperature may transmit temperature information to a laboratory; a laboratory may use such temperature information in the control of the operation of the instrument, in the oversight of the instrument, and in the analysis of data transmitted from the instrument.

In embodiments, a data acquisition device may be configured to acquire images from within the device, or within a portion of the data acquisition device. Such images may provide information about the position, condition, availability, or other information regarding components, reagents, supplies, or samples within the data acquisition device, and may provide information used in control of the operation of the data acquisition device. Such images may be useful for quality control. A data acquisition device that acquires images from within the device may transmit image information to a laboratory; a laboratory may use such image information in the control of the operation of the instrument, in the oversight of the instrument, and in the analysis of data transmitted from the instrument.

### Process Management Operations

As summarized above, aspects of the present disclosure include providing a process management operation such that the evaluation for the biological sample can be used for screening, diagnosis or treatment of the subject.

In some embodiments, the process management operation includes quality assurance and oversight of sample handling and data acquisition. This may be achieved through means to control for the quality of the raw data obtained from the sample acquisition site as well as determine the competency of the operator of the D2PA and/or data acquisition devices.

Quality assurance of the raw data may involve providing appropriate control or calibration methods in the D2PA device 104 such that the raw data can be interpreted properly despite variations and/or errors in sample handling. Significant errors due to, for example, the user, e.g., patient, handling of the sample, could be due to the sample collection method. A patient may not collect the correct volume of the sample, the collection may not be performed at the appropriate time, or the sample may not be handled in an appropriate manner, thus compromising the sample integrity. In some instances, the data acquisition device and/or the D2PA device may be configured to minimize the consequences of such errors by, for example, either alerting the patient to repeat the test or use calibration steps to compensate for errors. Calibration of the data acquisition device and/or the D2PA device may be performed at a manufacturing site. In some embodiments, calibration may involve using an internal calibration or a spike recovery method, as described in U.S. Pat. No. 7,635,594, which is incorporated herein by reference.

A calibration spike can be enabled by adding analyte to the antibody (or other solid phase capture agent) during manufacturing, and then drying, subsequently adding analyte to the appropriate detection area of a D2PA device during manufacturing (then drying), or adding analyte to a portion of assay buffer which is then routed to the appropriate detection area of a D2PA device.

Errors in measuring analyte concentrations could also be due to variability in the sample preparation phase. The primary cause of this type of errors is due to the user, e.g., patient, collecting an incorrect volume of sample or where the sample integrity has been compromised. Errors due to incorrect sampling volume can by corrected by a variety of means. One method is to measure the volume of the sample during a pre-processing step. If the measured volume is significantly different from the expected volume, the patient could be instructed to provide a new sample. This could be accomplished by, for example, the data acquisition device detecting an insufficient amount of fluid in the D2PA device. Alternatively, the analytical methods or algorithms on the data acquisition device could be recalibrated to compensate for the change in the sample volume. The recalibration could be using any of the standard calibration techniques or the modifications to the calibration process.

In one example of detecting an insufficient amount of sample volume, the D2PA device 104 may be lined with conductive elements spaced apart at known separations similar to the graduations on a measuring cylinder or jar. The location of each conductor can correspond to a specific sample volume. As fluid comes into contact with the conductor, the measured conductivity of that conductor would be markedly increased. By identifying the highest placed conductor that has undergone the conductivity change, the volume of the sample in the sample collection unit can be computed.

Alternatively, if the sample volume has to meet a minimum, a conductive element could be placed at the appropriate level in the sample receiving area. When the D2PA device is read by the data acquisition device, and if the conductivity of the sensor remains at the baseline level, it could be easily concluded that the patient has not provided the required sample volume. The patient could be given the appropriate feedback such as replacing the sample or replenishing it. Alternatively, a communication device or processor at the first site 120 could be informed of the issue and appropriate corrective measures taken. An alternative to the electrical sensing for the correct volume could be using known optical sensing means.

In some embodiments, the process management operation includes quality assurance of the data acquisition device and/or D2PA device 104. As described above, the D2PA and/or D2PA device may contain an identification mark, such as a barcode or RFID tag, which may convey relevant information such as, for example, the identity of a specific D2PA device type, date and location of manufacture, manufacturing lot number, expiration date, a unique number associated with a D2PA device, etc.

Other information encoded with the D2PA device 104 may include the refrigerator shelf life, the ambient temperature shelf life, the age of the device and the like. Alternatively, rather than including numerous elements of relevant information, a single piece of information, e.g., a lot number, may be included. The lot number may be any alphanumeric sequence or unique identifier that can be used to identify the D2PA device and associate relevant information with that D2PA device. For example, the lot number can be applied to a lookup table or any other type of computer database located within or connected to the data acquisition device or any other computing system that communicates with the data acquisition device. Using the lookup table or computer database, relevant shelf life or other such information can be associated with the lot number such that, based on the lot number, the refrigerator shelf life, the ambient temperature shelf life, the age of the D2PA device and the like can be determined.

The D2PA device 104 may have a finite refrigerator and ambient temperature shelf life. For example, the D2PA device may have a refrigerated usable lifetime in the range of, for example, about three months to three years, although the D2PA device could have any range of refrigerated usable lifetime. The D2PA device may have an ambient temperature usable lifetime in the range of, for example, about three days to three months, although the D2PA device can have any range of ambient temperature usable lifetime. Given that the D2PA device may have a finite refrigerator and ambient temperature shelf life, there may be a need to ensure that expired D2PA device (e.g., the D2PA device that have exceeded the refrigerated or ambient temperature shelf life) are not used.

In some embodiments a data acquisition device 102 may contain instructions, e.g., a medical app or other software, in a memory location, that contains instructions that allows the user, e.g., a patient or doctor attending to a patient, to use the data acquisition device to analyze, via a D2PA device, a biological sample collected from the patient. The software may be configured to provide information about the operation of the software and/or the data acquisition device to server, via any convenient communication interface, such as a networking interface on the data acquisition device, for quality assurance. The software may be updated to fix errors in the software code, add new functionalities, comply with new regulation, and the like.

In certain embodiments, a data acquisition device 102 may monitor its environment to provide quality assurance information. In embodiments, a data acquisition device may provide device environmental information to a laboratory. Device environmental information includes, e.g., temperature, humidity, time, status of components, error codes, air pressure (barometric pressure), and other information.

Thus, in some embodiments, monitoring, and reporting the results of such monitoring, of the data acquisition device environment may be part of the oversight of the operation of the data acquisition device and of the integrity of results and analysis. Such monitoring provides information, oversight, and control of quality regarding the data acquisition device and its output (of the condition, operation, results generated, and data transmitted). Such monitoring may include: measurement of air temperature; measurement of liquid (volume, temperature, mixing, etc.); monitoring of sample collection; imaging of a D2PA device to determine the position of a D2PA device in the data acquisition device; imaging of bubbles in liquids, if any; imaging of samples to assess sample volume, sample quality and presence or absence of conditions which may interfere with proper processing or analysis (such as hemolysis, lipemic, icteric conditions of a sample); feedback control and error detection of status and condition of motors on a pipette for control and oversight concerning accurate aspiration and dispensing of liquids; monitoring and control of the proper state of reagents; the running of control reactions at the same time as the sample analysis, or beforehand or subsequently; performance of replicate analysis of the sample to enhance precision; performing blank reads to control for possible changes in background signals in samples and for possible small fluctuations in sensor performance and in output of light sources.

In embodiments, monitoring, and reporting the results of such monitoring, may include calibration. Calibration of the devices, reagents, disposables, and of their manufacture and assembly may be part of the oversight of the operation of the data acquisition data acquisition device and of the integrity of results and analysis. During manufacturing, each device is calibrated to a set of controlled standards. During manufacturing, reagents and disposables (e.g., tips, cuvettes, and other elements) is calibrated to a set of controlled standards, and identification information related to each D2PA device containing these reagents and disposables includes information about these calibrations. Such calibration may include: each sensor and illumination source in a device is calibrated against a set of controlled standards, and provided in the information for each device, so that the resulting signals from all sensors across all data acquisition devices results in the same measurement; each camera and each illumination source is characterized, including a flat field correction; during manufacturing, each of lot reagents is calibrated such that any change in potency of the reagents still results in the same analytic result. Thus, since each sample is analyzed by processing on the device (for which such component and device specific calibration information is known) the raw data resulting from sample processing can be calibrated and corrected according to the information and calibrations for each reagent, and each device and its components. Such oversight and calibration insures the integrity of the results obtained, and thus also provides for the integrity of analysis of such results. The raw data from a data acquisition device is analyzed by utilizing the device-specific calibration and the reagent lot-specific calibration to arrive at the result. Each result arrived at in this way is thus accurate, precise, and reliable, and may be compared with analogous results obtained from other samples in the same instrument, and with other instruments and samples, reducing variance and errors and allowing for better analysis and better confidence in diagnoses and inferences drawn from the analysis of samples.

Quality control runs may be performed on data acquisition and/or D2PA devices on a periodic basis, e.g., as overseen by CLIA, to ensure that the devices are still performing within specifications. If discrepancies are found, reagents and/or devices may be determined to need recalibration. Devices can be recalibrated in the field by running defined protocols, which may or may not require inserting a calibration cartridge into the device. Reagents may be recalibrated by generating a standard curve using the same lot of reagents and deriving a calibration function. Such reagent recalibration can performed on any one of the devices and can be applied to all devices.

Transmission of data acquisition device environmental information to a laboratory is useful for the oversight and control of the device, including being useful for the oversight and control of the operation of the device. Transmission of data acquisition device environmental information to a laboratory is useful for maintaining the integrity of the operation and control of the device, quality control of the operation and control of the device, and for reducing variation or error in the data collection and sample processing performed by the device. Device environmental information may be used, for example, by a laboratory to modify, correct, or update a protocol or other instruction or command to a device. Device environmental information may be used, for example, by a laboratory to modify, correct, or update an analysis of data received from a device. For example, transmission of temperature information to a laboratory is useful for the oversight and control of the data acquisition device, and is useful in the analysis by the laboratory of data provided by the data acquisition device to the laboratory.

The process management operation may further include an assessment of the competency of the operator, e.g., subject or doctor, of the data acquisition and/or D2PA devices. In some embodiments, the assessment of the operator is achieved by a compliance monitoring system. In general terms, an operator's competency and compliance assessment may involve evaluation of an individual's knowledge, skills, and correct practice of required processes and procedures. Typically, competency assessment programs include evaluating the competency of all testing personnel and assuring that the staff maintains their competency to perform test procedures and report result promptly, accurately, and proficiently. Examples of methods and systems for assessing competency of an operator in a point-of-care testing system is described, e.g., in U.S. App. Pub. No. 20140278832, which is incorporated herein by reference.

In some embodiments, the process management process includes oversight of the analysis performed on the raw data at, e.g., an authorized analytical facility. An authorized analytical facility is typically subject to oversight or regulation. For example, a laboratory may have oversight by a board-certified entity (which may include one or more board-certified personnel). In some embodiments, oversight can include validating one or more clinical test. Oversight may also include assessing the performance of, correcting, calibrating, running controls, running replicates, adjusting, or analyzing one or more clinical test. Oversight can include evaluation of one or more sets of data to provide a quality control for a clinical test. The authorized analytical facility can have one or more qualified person providing the oversight. For example, one or more pathologist or other health care professional may review data and/or analysis that is processed by the facility. At an authorized analytical facility, a trained pathologist or other certified health care professional may provide oversight. In some instances, the certified health care professional providing oversight may be one or more of the following: a doctor certified in pathology, a doctor with laboratory training or experience in the specialty areas of service for which the health care professional is responsible, or an individual with experience or laboratory training in the specialty.

The oversight may further include the certified health care professional 140 who may establish the procedures and rules in the laboratory, deal with problems that arise, and/or train/evaluate the lab personnel. Oversight may also include selecting test methodology, verifying test procedures and establishment of laboratory's test performance characteristics, enrollment in participation in a U.S. Human and Health Services (HHS) approved proficiency testing program, establishing a quality control program appropriate for the testing performed, establishing the parameters for acceptable levels of analytic performance, ensuring that those levels are maintained throughout the entire testing process, resolving technical problems and ensuring that remedial actions are taken when test systems deviate from the established performance specifications, ensuring patient test results are not reported until all corrective actions have been taken, identifying training needs and assuring that each individual performing tests receives regular in-service training and education, evaluating the competency of all testing personnel and assuring that the staff maintain their competency to perform test procedures (e.g., also procedures for evaluation of the staff: direct observation of routine test performance, monitoring the recording/reporting of results, review of intermediate test results, records, etc, observation of performance of instrument maintenance, assessment of test performance, assessment of problem solving skills), and/or evaluating and documenting the performance of individuals responsible for moderate complexity testing (e.g., semiannually during the first year; thereafter, at least annually unless test methodology or instrumentation changes). Oversight may include reviewing and/or verifying functionality of laboratory procedures or devices, and/or validity of data collected and/or generated. The oversight may assure the quality of the rest and/or put the data into a condition upon which a health care professional can rely upon it to provide a screening, diagnosis, treatment, including but not limited to prophylactic treatment. Oversight may include reviewing a test empirically. Oversight may include one or more, two or more, or any of the number of items described elsewhere herein.

In some instances, the oversight may be provided by an oversight software program rather than the certified health care professional 140. In some instances, one, two or more of the types of oversight provided may be implemented by an oversight software program. A combination of an oversight software program and health care professional may be employed to provide oversight. In some instances, one, two or more of the types of oversight may be implemented by a health care professional over a software program. For example, the health care professional may determine the procedures and rules associated with the software program. In some instances, the software program may be self-learning. The software program may access an increasing pool of data and/or evolving rules or procedures.

In some embodiments, the oversight software program may be provided on a computing device. The oversight software program may be provided at a sample collection site, on or off the computing device. The software program may be provided at a laboratory, such as an authorized analytical facility. In some instances, the device may receive updates to the oversight software program. The updates may or may not be provided by the laboratory. The oversight software may be stored in a memory, and may include computer readable media comprising code, instructions, or logics that may be capable of executing a step.

In some instances, the oversight software may include one or more algorithm that may review a qualitative and/or quantitative evaluation of the sample that may be performed. The oversight software program may look for outliers, may determine whether the qualitative and/or quantitative evaluation was properly performed, may perform one or more comparison with records or data points, may perform statistical analysis of the evaluation, or any other oversight action as described elsewhere herein. The oversight software may be able to perform one or more calibrations and/or diagnostics.

Suitable methods and systems for providing oversight to point-of-care testing is described in, e.g., U.S. App. Pub. No. 20140057255, which is incorporated herein by reference.

A health care professional of an authorized analytical facility may receive and/or view data. A health care professional of an authorized analytical facility may be affiliated with or associated with the authorized analytical facility. In some instances, the health care professional may be employed by or under contract with the authorized analytical facility. The health care professional may be located at the authorized analytical facility, may be located remotely from the authorized analytical facility, or in another analytical facility (e.g., hospital, center of excellence, specialized leading path/group). In some instances, the health care professional is not required to be on-site at all times while testing is performed, or when data is received at an authorized analytical facility, but may be available on an as needed basis to provide consultation. The health care professional may be accessible to provide on-site, telephone and/or electronic consultation.

The health care professional 140 providing oversight may be a different individual from or the same individual as the health care professional that may receive a report from the authorized analytical facility for diagnosing, treating, monitoring, or preventing a disease for the subject. For example, a pathologist of an authorized analytical facility may be a different individual from a prescribing physician of the subject. A health care professional of authorized analytical facility may be a reviewing health care professional or an overseeing health care professional. The health care professional who may receive the report may be the health care professional who has ordered the test that the subject has undertaken. A different health care professional may provide analysis, and a different health care professional may provide oversight. Alternatively, the same health care professional may provide both analysis and oversight.

### Additional Embodiments

In certain embodiments, the present method includes generating a report including the evaluation of the raw data. The report may contain any suitable information that is pertinent to the source from which the analyte was obtained. In some instances, the report may include: light data, including light intensity, wavelength, polarization, and other data regarding light, e.g., output from optical detectors such as photomultiplier tubes, photodiodes, charge-coupled devices, luminometers, spectrophotometers, cameras, and other light sensing components and devices, including absorbance data, transmittance data, turbidity data, luminosity data, wavelength data (including intensity at one, two, or more wavelengths or across a range of wavelengths), reflectance data, refractance data, birefringence data, polarization, and other light data; image data, e.g., data from digital cameras; the identifier information associated with the D2PA device used to acquire the data; the processed data, as described above, etc. The report may represent qualitative or quantitative aspects of the sample.

The analytical facility 122, e.g., laboratory, and/or data acquisition device may provide a report to the subject. The report provided to the subject may be the same as or different from the report provided to a health care professional 140. The report provided to the health care professional may have more detail or vice versa. The formats between the reports provided to the subject and the health care professional may or may not vary. Alternatively, the laboratory and/or device does not provide a report to the subject. The subject may receive information based on the report from the health care professional. A device or laboratory can directly provide a lab report automatically to a consumer upon a test being performed and/or analysis being done, or being sent to a physician for review and/or after the physician's review.

In certain aspects, the report may indicate to the subject the presence or absence of an analyte, the concentration of an analyte, the presence or absence of a secondary condition known to be correlated with the presence or level of the analyte, the probability or likelihood of a secondary condition known to be correlated with the presence or level of the analyte, the likelihood of developing a secondary condition known to be correlated with the presence or level of the analyte, the change in likelihood of developing a secondary condition known to be correlated with the presence or level of the analyte, the progression of a secondary condition known to be correlated with the presence or level of the analyte, etc. The secondary condition known to be correlated with the presence or level of the analyte may include a disease or health condition for a diagnostic sample. In certain embodiments, the report contains instructions urging or recommending the user to take action, such as seek medical help, take medication, stop an activity, start an activity, etc. The report may include an alert. One example of an alert may be if an error is detected on the device, or if an analyte concentration exceeds a predetermined threshold. The content of the report may be represented in any suitable form, including text, graphs, graphics, animation, color, sound, voice, and vibration.

In certain embodiment, the report provides an action advice to the user of the subject device, e.g., a mobile phone. The advices will be given according to the test data by the devices (e.g. detectors plus mobile phone) together with one or several data sets, including but not limited to, the date preloaded on the mobile devices, data on a storage device that can be accessed, where the storage device can be locally available or remotely accessible.

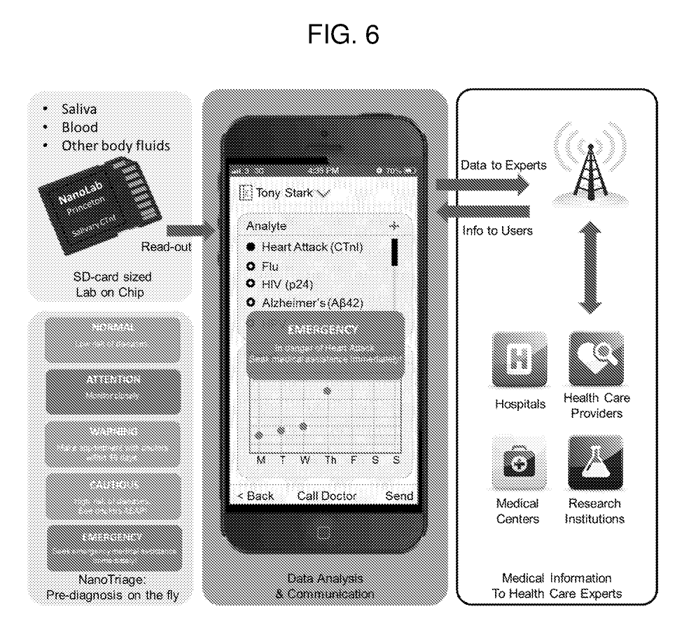

In certain embodiments, each of the advices above has its own color in scheme in the mobile phone displays. One example is given in FIG. 6.

The report may have a format that may enable a viewer of the report to rely on the report to make a medical determination. The analytical facility 122, e.g., laboratory, may transmit the report to a health care professional 140 (or laboratory director). In some embodiments, a pathologist, other health care professional, or other qualified person may review the report prior to transmitting the report to the health care professional. A reviewing health care professional may review the report or qualitative and/or quantitative evaluation useful for generating the report prior to transmission to an ordering health care professional. Review or oversight may occur of the analyzed data and/or report at the laboratory. The health care professional who receives the report may or may not rely on the report for screening, diagnosis, treatment and/or disease prevention of the subject.

Any transmission of data and/or reports may incorporate the use of a cloud computing infrastructure. The sending party may provide the data to or have the data on a cloud computing infrastructure. The receiving party and/or parties (e.g., health care professional or patient) may access the cloud computing infrastructure. The cloud computing infrastructure may be provided on the sending party side and/or the receiving party side. Alternatively, traditional fixed data storage techniques may be employed.

### Systems

Also provided herein is a system that finds use in performing the present method of analyzing a biological sample collected from a subject, as described above. An implementation of the present system may include a) a communication device at a first site wherein the communication device receives data transmitted from a data acquisition device at a second site, wherein the data acquisition device is configured to i) read an output signal from a D2PA device, as described herein, and ii) transmit data containing the raw data to the first site, and b) a processor at the first site, wherein the processor analyzes and generates an evaluation of the data

A communication device and a processor may be located at an analytical facility 122, e.g., a laboratory, that is remote from the location of a data acquisition device and a D2PA device, e.g., a sample collection site 100. As such the first site and the second site may be remote. In some embodiments, the first site and second site are the same site, e.g., in the same room.

One or more communication units may be provided at the analytical facility 122, e.g., laboratory. The laboratory may be at the same location as or different location from, or may actually be the same as the sample collection or processing center or provider or hospital office/location. Any description herein of the laboratory may apply to any other locations provided herein and vice versa. The communication unit may be configured to receive data from a data acquisition device. The communication unit may receive data relating to a sample of a subject from the data acquisition device at a sample collection site. The communication unit may receive information about the subject from the data acquisition device and/or the sample collection site. The communication unit may receive identifying information about the subject. The communication unit may receive information from the data acquisition device and/or any other machine (e.g., biometric devices, mobile devices) or entity associated with the sample collection site.

The communication unit may be configured to transmit data to a data acquisition device and/or any other machine or entity associated with the sample collection site. In some embodiments, the communication unit may provide one or more protocol to the data acquisition device. The communication may provide the protocol in addition to receiving data. The protocol may effect the running of the clinical test on the data acquisition device. The protocol may effect the detection of the presence and/or concentration of an analyte at the D2PA device. Any description of detection and/or analysis relating to the presence and/or concentration of an analyte may include and/or be applied to assessing a disease condition. The protocol may effect the pre-processing of raw data and/or analysis of data at the data acquisition device.

The communication unit may permit two-way communication unit between the sample collection site and the analytical facility 122, e.g., laboratory. The communication unit may permit two-way communication between a sample processing device at a sample collection site or in or on a subject, and a processor at the laboratory. In some embodiments, one or more protocol may be sent to a device based on data sent by the data acquisition device. The data sent by the data acquisition device may include subject identifying information, information based on signals generated and/or detected relating to the sample or reactions, data acquisition device identification information, D2PA device identification information, or any other information sent from the data acquisition device. Data may be collected from the device depending on protocols provided to the data acquisition device. The protocols may govern the type of data that is collected and the actions performed by the data acquisition device. In some embodiments, one, two, or more subsequent sets of protocols may be sent to a data acquisition device based on data collected from the data acquisition device. The data from the data acquisition device may provide feedback which may govern further actions to be taken by the data acquisition device, dictated by the protocols.

In alternate embodiments of the invention, the analytical facility 122, e.g., laboratory need not send protocols to the data acquisition device. The protocols may be stored locally on the data acquisition device. Alternatively, the system may provide protocols to the data acquisition device. The protocols may be provided from an entity external to the data acquisition device.

The communication unit may be configured to transmit data to a health care professional 140. In some embodiments, the communication unit may transmit a report or analysis generated based on data relating to the sample. The communication unit may be in communication with a network device used by the health care professional. For example, the communication unit may be capable of communicating with a computer, tablet, or mobile device of the health care professional.

In some embodiments, the processor provides one or more process management operations, as described above, such that the evaluation can be used for screening, diagnosis, or treatment of the subject.

A processor may be configured to generate a report for a health care professional 140. The processor may be on a server side with a software performing the processing. The processor may generate the report based on data received from the data acquisition device 102 or may provide oversight or analysis. The processor may perform qualitative and/or quantitative evaluation of the sample. In some embodiments, the processor may compare data received from the data acquisition device with a threshold value. The threshold value may be for one or more analyte. Said comparison may include a comparison of whether a data value is greater than, equal to, or less than a threshold value. The comparison may include whether the data value is qualitatively and/or quantitatively the same as the threshold value. The comparison may include one or more forms of statistical or physiological analysis of the data in relation to one or more stored values. Examples may include best-fit analysis, and/or analysis such as curve fitting, extrapolation, interpolation, regression analysis, least squares, mean calculations, multivariate, simulation analysis, or variation calculations. The processor may analyze the data received from the data acquisition device. The processor may be configured to perform one or more steps for statistical analysis of the data.

In some embodiments, a threshold value may refer to a single value. The threshold value may be a numerical value or an alphanumeric value. The threshold value may be a string or any other form of data. The threshold value may refer to a range of values and/or set of values. A threshold value may refer to a single value or a plurality of values. A plurality of values may fall within one or more continuous spectrum. Alternatively, the plurality of values may be discrete.

Examples of threshold ranges may include 1-100 units, or 5-10 units, and examples of threshold sets may include values falling within a list selected from 1 unit, 3 units, 5 units, 8 units, 13 units, 20 units, or 50 units. A unit may refer to any dimension or measureable quantity. Such values are provided by way of example only. In some instances, the processor may compare one or more image, video, or audio file or other data. The processor may make such comparisons against one or more reference image, video, or audio file or other data. An algorithm may be capable of evaluating one or more feature of the files or other data. In some instances, the processor may automatically sort the files for viewing by a health care professional.

The processor may be able to access one or more data storage unit, which may contain stored information. The stored information may include the threshold value for one or more analyte. The threshold value may be useful for determining the presence or concentration of the one or more analyte. The threshold value may be useful for detecting situations where an alert may be useful. The data storage unit may include any other information relating to sample preparation or clinical tests that may be run on a sample. The data storage unit may include records or other information that may be useful for generating a report for a health care professional. The data storage units may also be capable of storing computer readable media which may include code, logic, or instructions for the processor to perform one or more step.

In some embodiments, a data storage unit may be provided at the analytical facility 122, e.g., laboratory. The processor may be able to access the local data storage unit. In another embodiment, the data storage unit may be provided remote to the laboratory. For example, the data storage unit may be provided at a sample collection site 100 or with a health care professional 140. The data storage unit may be provided on the data acquisition device. Alternatively, the data storage unit may be provided at any other location. Any combination of data storage unit locations may be utilized by the processor. For example, the processor may access data storage units that may be provided at the laboratory and external to the laboratory.

In some embodiments, the data storage units may be electronic medical records (EMR) or EMR databases. The data storage units may contain information associated with a subject. The information associated with the subject may include medical records of the subject, health history of the subject, identifying information associated with the subject, payment information associated with the subject, or any other information associated with the subject. The data storage units may be payer databases. The data storage units may include information associated with a payer, such as a health insurance company or governmental payer. Such information may include treatment records, insurance records, or financial information associated with the subject.

The laboratory may have an output unit which may display or transmit the report to the health care professional. The output unit may be a video display. Alternatively, the output unit may be a communication unit. In one example, the output unit may be a touchscreen. The touchscreen may have an intrinsic imaging capability through built-in sensors, which may include LEDs or other light sources.

In some embodiments, the processor, communication unit, and data storage unit may be provided on the same machine. Alternatively, two or more of the processor, communication unit, and data storage unit may be provided on the same machine. The machine may be a computer, or any other network device as described elsewhere herein. Two or more of the processor, communication unit, and data storage may be located on a laboratory-located computer. Alternatively, the processor, communication unit, and data storage may all be located on different machines. In some instances, multiple processors, communication units, and data storage units may be provided that may be distributed over one or a plurality of machines.

### Utility

The present method and system find use in a variety of different applications where point-of-care screening, diagnosis, and/or treatment of a subject are desired. For example, the subject method finds use in screening, diagnosis, and/or treatment of a subject at a subject's home, doctor's office, emergency room, or hospital bedside, through the detection of proteins, peptides, nucleic acids, and the like. In certain embodiments, the present method and system finds use in the detection of nucleic acids, proteins, or other biomolecules in a sample without the need for access to sophisticated equipment at the site of sample collection. For example, the methods may be used in the rapid, clinical detection of two or more disease biomarkers in a biological sample, e.g., as may be employed in the diagnosis of a disease condition in a subject, or in the ongoing management or treatment of a disease condition in a subject, etc. As described above, communication to a physician or other health-care provider may better ensure that the physician or other health-care provider is made aware of, and cognizant of, possible concerns and may thus be more likely to take appropriate action.

The applications of the method and system include, but are not limited to, (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., virus, fungus and bacteria from environment, e.g., water, soil, or biological samples, e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g., during synthesis or purification of pharmaceuticals.

In certain embodiments, the present method and system find use in detecting biomarkers. In some cases, the subject systems and methods may be used to detect the presence or absence of particular biomarkers, as well as an increase or decrease in the concentration of particular biomarkers in blood, plasma, serum, or other bodily fluids or excretions, such as but not limited to urine, blood, serum, plasma, saliva, semen, prostatic fluid, nipple aspirate fluid, lachrymal fluid, perspiration, feces, cheek swabs, cerebrospinal fluid, cell lysate samples, amniotic fluid, gastrointestinal fluid, biopsy tissue, and the like.

The presence or absence of a biomarker or significant changes in the concentration of a biomarker can be used to diagnose disease risk, presence of disease in an individual, or to tailor treatments for the disease in an individual. For example, the presence of a particular biomarker or panel of biomarkers may influence the choices of drug treatment or administration regimes given to an individual. In evaluating potential drug therapies, a biomarker may be used as a surrogate for a natural endpoint such as survival or irreversible morbidity. If a treatment alters the biomarker, which has a direct connection to improved health, the biomarker can serve as a surrogate endpoint for evaluating the clinical benefit of a particular treatment or administration regime. Thus, point-of-care diagnosis and treatment based on the particular biomarkers or panel of biomarkers detected in an individual are facilitated by the subject method. Furthermore, the early detection of biomarkers associated with diseases is facilitated by the high sensitivity of the subject devices and systems, as described above. Due to the capability of detecting multiple biomarkers with a mobile device, such as a smartphone, combined with sensitivity, scalability, and ease of use, the presently disclosed systems and methods find use in portable and point-of-care or near-patient molecular diagnostics.

In certain embodiments, the subject method finds use in detecting biomarkers for a disease or disease state. In certain instances, the subject method finds use in detecting biomarkers for the characterization of cell signaling pathways and intracellular communication for drug discovery and vaccine development. For example, the subject systems and methods may be used to detect and/or quantify the amount of biomarkers in diseased, healthy or benign samples. In certain embodiments, the subject method finds use in detecting biomarkers for an infectious disease or disease state. In some cases, the biomarkers can be molecular biomarkers, such as but not limited to proteins, nucleic acids, carbohydrates, small molecules, and the like.

The subject method find use in point-of-care diagnostic assays, such as, but not limited to, the following: detecting and/or quantifying biomarkers, as described above; screening assays, where samples are tested at regular intervals for asymptomatic subjects; prognostic assays, where the presence and or quantity of a biomarker is used to predict a likely disease course; stratification assays, where a subject's response to different drug treatments can be predicted; efficacy assays, where the efficacy of a drug treatment is monitored; and the like.

In some cases, the D2PA may be employed to detect a biomarker that is present at a low concentration. For example, the D2PA may be used to detect cancer antigens in a readily accessible bodily fluids (e.g., blood, saliva, urine, tears, etc.), to detect biomarkers for tissue-specific diseases in a readily accessible bodily fluid (e.g., a biomarkers for a neurological disorder (e.g., Alzheimer's antigens)), to detect infections (particularly detection of low titer latent viruses, e.g., HIV), to detect fetal antigens in maternal blood, and for detection of exogenous compounds (e.g., drugs or pollutants) in a subject's bloodstream, for example.

The following table provides a list of protein biomarkers that can be detected using the present method employing a D2PA device (when used in conjunction with an appropriate monoclonal antibody), and their associated diseases. One potential source of the biomarker (e.g., “CSF”; cerebrospinal fluid) is also indicated in the table. In many cases, the subject biosensor can detect those biomarkers in a different bodily fluid to that indicated. For example, biomarkers that are found in CSF can be identified in urine, blood or saliva.

| Marker | disease |
|---|---|
| Aβ42, amyloid beta-protein (CSF) | Alzheimer's disease. |
| fetuin-A (CSF) | multiple sclerosis. |
| tau (CSF) | niemann-pick type C. |
| secretogranin II (CSF) | bipolar disorder. |
| prion protein (CSF) | Alzheimer disease, prion disease |
| Cytokines (CSF) | HIV-associated neurocognitive disorders |
| Alpha-synuclein (CSF) | parkinsonian disorders (neuordegenerative |
| | disorders) |
| tau protein (CSF) | parkinsonian disorders |
| neurofilament light chain (CSF) | axonal degeneration |
| parkin (CSF) | neuordegenerative disorders |
| PTEN induced putative kinase 1 (CSF) | neuordegenerative disorders |
| DJ-1 (CSF) | neuordegenerative disorders |
| leucine-rich repeat kinase 2 (CSF) | neuordegenerative disorders |
| mutated ATP13A2 (CSF) | Kufor-Rakeb disease |
| Apo H (CSF) | parkinson disease (PD) |
| ceruloplasmin (CSF) | PD |
| Peroxisome proliferator-activated receptor | PD |
| gamma coactivator-1 alpha (PGC-1α) (CSF) | |
| transthyretin (CSF) | CSF rhinorrhea (nasal surgery samples) |
| Vitamin D-binding Protein (CSF) | Multiple Sclerosis Progression |
| proapoptotic kinase R (PKR) and its | AD |
| phosphorylated PKR (pPKR) (CSF) | |
| CXCL13 (CSF) | multiple sclerosis |
| IL-12p40, CXCL13 and IL-8 (CSF) | intrathecal inflammation |
| Dkk-3 (semen) | prostate cancer |
| p14 endocan fragment (blood) | Sepsis: Endocan, specifically secreted by |
| | activated-pulmonary vascular endothelial |
| | cells, is thought to play a key role in the |
| | control of the lung inflammatory reaction. |
| Serum (blood) | neuromyelitis optica |
| ACE2 (blood) | cardiovascular disease |
| autoantibody to CD25 (blood) | early diagnosis of esophageal squamous cell |
| | carcinoma |
| hTERT (blood) | lung cancer |
| CAI25 (MUC 16) (blood) | lung cancer |
| VEGF (blood) | lung cancer |
| sIL-2 (blood) | lung cancer |
| Osteopontin (blood) | lung cancer |
| Human epididymis protein 4 (HE4) (blood) | ovarian cancer |
| Alpha-Fetal Protein (blood) | pregnancy |
| Albumin (urine) | diabetics |
| albumin (urine) uria | albuminuria |
| microalbuminuria | kidney leaks |
| AFP (urine) | mirror fetal AFP levels |
| neutrophil gelatinase-associated lipocalin (NGAL) | Acute kidney injury |
| (urine) | |
| interleukin 18 (IL-18) (urine) | Acute kidney injury |
| Kidney Injury Molecule -1 (KIM-1) (urine) | Acute kidney injury |
| Liver Fatty Acid Binding Protein (L-FABP) (urine) | Acute kidney injury |
| LMP1 (saliva) | Epstein-Barr virus oncoprotein |
| | (nasopharyngeal carcinomas) |
| BARF1 (saliva) | Epstein-Barr virus oncoprotein |
| | (nasopharyngeal carcinomas) |
| IL-8 (saliva) | oral cancer biomarker |
| carcinoembryonic antigen (CEA) (saliva) | oral or salivary malignant tumors |
| BRAF, CCNI, EGRF, FGF19, FRS2, GREB1, and | Lung cancer |
| LZTS1 (saliva) | |
| alpha-amylase (saliva) | cardiovascular disease |
| carcinoembryonic antigen (saliva) | Malignant tumors of the oral cavity |
| CA 125 (saliva) | Ovarian cancer |
| IL8 (saliva) | spinalcellular carcinoma. |
| thioredoxin (saliva) | spinalcellular carcinoma. |
| beta-2 microglobulin levels - monitor activity of | HIV |
| the virus (saliva) | |
| tumor necrosis factor-alpha receptors - monitor | HIV |
| activity of the virus (saliva) | |
| CA15-3 (saliva) | breast cancer |

The health conditions that may be diagnosed or measured by the present point-of-care method, device and system include, but are not limited to: chemical balance; nutritional health; exercise; fatigue; sleep; stress; prediabetes; allergies; aging; exposure to environmental toxins, pesticides, herbicides, synthetic hormone analogs; pregnancy; menopause; and andropause.

The subject method and system also finds use in validation assays. For example, validation assays may be used to validate or confirm that a potential disease biomarker is a reliable indicator of the presence or absence of a disease across a variety of individuals. The short assay times for the subject method may facilitate an increase in the throughput for screening a plurality of samples in a minimum amount of time.

As described above, the present method and system provides a way for a user, e.g., a patient or doctor, to analyze a biological sample collected from a subject without requiring a laboratory setting. In comparison to the equivalent analytic research laboratory equipment, the subject method provides comparable analytic sensitivity in a portable, hand-held system. In some cases, the mass and operating cost are less than the typical stationary laboratory equipment. In addition, the subject method can be utilized in a home setting for over-the-counter home testing by a person without medical training to detect one or more analytes in samples. The subject method may also be utilized in a clinical setting, e.g., at the bedside, for rapid diagnosis or in a setting where stationary research laboratory equipment is not provided due to cost or other reasons.

In certain embodiments, relative levels of nucleic acids in two or more different nucleic acid samples may be obtained using the above methods, and compared. In these embodiments, the results obtained from the above-described methods are usually normalized to the total amount of nucleic acids in the sample (e.g., constitutive RNAs), and compared. This may be done by comparing ratios, or by any other means. In particular embodiments, the nucleic acid profiles of two or more different samples may be compared to identify nucleic acids that are associated with a particular disease or condition.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

1. A method of analyzing a biological sample collected from a subject, comprising:
a) receiving, at a first site, data transmitted from a data acquisition device at a second site, wherein the data acquisition device is configured to:
i) read an output signal from a disk-coupled dots-on-pillar antenna array (D2PA) device to generate raw data that is representative of the biological sample, wherein the D2PA device comprises a D2PA configured to:
bind an analyte of interest; and
generate an output signal upon contacting the biological sample, wherein the output signal is representative of the sample; and,
wherein the D2PA device is configured to:
receive the biological sample; and
contact the biological sample with the D2PA; and
ii) transmit data comprising the raw data to the first site; and
b) analyzing the data at the first site to generate an evaluation of the data.

2. The method of claim 1, wherein the data acquisition device is a mobile device.

3. The method of any of claim 1, wherein the first site and the second site are the same.

4. The method of any of claim 1, wherein the first site is remote from the second site.

5. The method of claim 1, wherein the method comprises generating a report comprising the evaluation of the data.

6. The method of claim 5, wherein the method comprises transmitting the report from the first site to a report display device, wherein the report display device is configured to receive and display the report.

7. The method of claim 6, wherein the report display device is at a second site that is remote from the first site.

8. The method of claim 6, wherein the report display device is the same device as the data acquisition device.

9. The method of claim 1, wherein the output signal comprises luminescence.

10. The method of claim 9, wherein the raw data is luminescence data that is representative of the sample.

11. The method of claim 1, wherein the raw data is representative of the amount of the analyte in the sample.

12. The method of claim 1, wherein the data acquisition device is configured to read an output signal from the sample acquisition device by acquiring an image of the D2PA to generate the raw data.

13. The method of claim 1, wherein the D2PA device is a microfluidic device or a micro titer plate.

14. The method of claim1, wherein the D2PA comprises a binding agent that specifically binds to the analyte of interest.

15. The method of claim 14, wherein the binding agent is an antibody or a nucleic acid.

16. The method of claim 1, wherein the first site comprises an authorized analytical facility and wherein the D2PA device is configured to transmit the data to the authorized analytical facility.

17. The method of claim 16, wherein the authorized analytical facility is a Clinical Laboratory Improvement Amendments (CLIA)-compliant laboratory.

18. The method of claim 1, wherein the second site is the subject's home, a health assessment location, or a health treatment location.

19. The method of claim 1, wherein the evaluation can be used by a health care professional for screening, diagnosis, or treatment of the subject.

20. The method of claim 1, wherein the evaluation can be used by the subject for diagnosis of the subject.

21. The method of claim 1, wherein the method comprises:
c) providing a process management operation such that the evaluation can be used for screening, diagnosis, or treatment of the subject.

22. The method of claim 21, wherein the process management operation comprises one or more of:
i) quality assurance of the raw data;
ii) quality assurance of the data acquisition device and/or D2PA device;
iii) oversight of the use of the data acquisition device and/or D2PA device;
iv) oversight of the analysis performed on the data to generate the evaluation; and
v) authorization of access to the evaluation and/or data by the health care professional.

23. A method of analyzing a biological sample, comprising:
a) transmitting to a first site, using a data acquisition device at a second site, data comprising raw data that is representative of a biological sample collected from a subject, wherein the raw data comprises a readout from a D2PA device comprising a D2PA, wherein the readout is obtained by reading an output signal from the D2PA device with the data acquisition device, wherein the D2PA device is configured to:
receive the biological sample; and
contact the received sample with the D2PA,
wherein the D2PA is configured to:
bind an analyte of interest; and
generate the output signal upon contacting the biological sample, wherein the output signal is representative of the sample,
wherein the first site comprises:
a communication device that receives data transmitted from the data acquisition device; and
a processor that analyzes and generates an evaluation of the data; and
b) receiving a report comprising an evaluation of the data.

24. The method of claim 23, wherein the data acquisition device is a mobile device.

25. The method of claim 23, wherein the first site and the second site are the same.

26. The method of claim 23, wherein the first site is remote from the second site.

27. The method of claim 23, wherein the raw data is representative of the amount of the analyte in the sample.

28. The method of claim 27, wherein the evaluation of the data comprises:
the amount of the analyte of interest in the sample; and
a range of values for the analyte in an individual free of or at low risk of being diagnosed with a disease or a health condition,
wherein the amount of the analyte relative to the range of values is diagnostic of the disease or health condition.

29. The method of claim 23, wherein the evaluation of the data comprises a diagnosis for a disease or a health condition provided by a health care professional.

30. The method of claim 23, wherein the evaluation of the data comprises a recommendation to seek medical attention.

31. The method of claim 23, wherein the D2PA device is a microfluidic device or a micro titer plate.

32. The method of claim 23, wherein the D2PA comprises a binding agent that specifically binds to the analyte of interest.

33. The method of claim 23, wherein the first site comprises an authorized analytical facility and wherein the D2PA device is configured to transmit the data to the authorized analytical facility.

34. The method of claim 33, wherein the authorized analytical facility is a Clinical Laboratory Improvement Amendments (CLIA)-compliant laboratory.

35. The method of claim 23, wherein the second site is the subject's home, a health assessment location, or a health treatment location.

36. The method of claim 23, wherein the evaluation can be used by a health care professional for screening, diagnosis, or treatment of the subject.

37. The method of claim 23, wherein the evaluation can be used by the subject for diagnosis of the subject.

38. The method of claim 23, wherein the processor provides a process management operation such that the evaluation can be used for screening, diagnosis, or treatment of the subject.

39. The method claim 38, wherein the process management operation comprises one or more of:
i) quality assurance of the raw data;
ii) quality assurance of the data acquisition device and/or D2PA device;
iii) oversight of the use of the data acquisition device and/or D2PA device;
iv) oversight of the analysis performed on the data to generate the evaluation; and
v) authorization of access to the evaluation and/or data by the health care professional.

40. A system for analyzing a biological sample collected from a subject, comprising:
a) a communication device at a first site, wherein the communication device receives data transmitted from a data acquisition device at a second site, wherein the data acquisition device is configured to:
i) read an output signal from a D2PA device comprising a disk-coupled dots-on-pillar antenna array (D2PA) to generate raw data that is representative of the biological sample, wherein the D2PA is configured to:
bind an analyte of interest; and
generate an output signal upon contacting the biological sample, wherein the output signal is representative of the sample;
and wherein the D2PA device is configured to:
receive the biological sample; and
contact the received biological sample with the D2PA; and
ii) transmit data comprising the raw data to the first site;
b) a processor at the first site, wherein the processor analyzes and generates an evaluation of the data.

41. The system of claim 40, wherein the data acquisition device is a mobile device.

42. The system of claim 40, wherein the first site and the second site are the same.

43. The system of claim 40, wherein the second site is remote from the first site.

44. The system of claim 40, wherein the processor is configured to generate a report comprising the evaluation of the data.

45. The system of claim 40, wherein the raw data is luminescence data that is representative of the sample.

46. The system of claim 40, wherein the raw data is representative of the amount of the analyte in the sample.

47. The system of claim 40, wherein the first site comprises an authorized analytical facility and wherein the D2PA device is configured to transmit the data to the authorized analytical facility.

48. The system of claim 47, wherein the authorized analytical facility is a Clinical Laboratory Improvement Amendments (CLIA)-compliant laboratory.

49. The system of any of claim 40, wherein the second site is the subject's home, a health assessment location, or a health treatment location.

50. The system of claim 40, wherein the evaluation can be used by a health care professional for screening, diagnosis, or treatment of the subject.

51. The system of claim 40, wherein the evaluation can be used by the subject for diagnosis of the subject.

52. The system of claim 40, wherein the processor provides a process management operation such that the evaluation can be used for screening, diagnosis, or treatment of the subject.

53. The system of claim 52, wherein the process management operation comprises one or more of:
i) quality assurance of the raw data;
ii) quality assurance of the data acquisition device and/or D2PA device;
iii) oversight of the use of the data acquisition device and/or D2PA device;
iv) oversight of the analysis performed on the data to generate the evaluation; and
v) authorization of access to the evaluation and/or data by the health care professional.
